SEQUENCE LISTING (6 sequences)

SEQ ID NO: 1 - DNA, 21 bp, Artificial Sequence
  PCR primer
gacattgacatccactttgcc

SEQ ID NO: 2 - DNA, 18 bp, Artificial Sequence
  PCR primer
gggagggaaacttggagg

SEQ ID NO: 3 - DNA, 21 bp, Artificial Sequence
  Oligonucleotide probe
gctgaaaggaagactcagagg

SEQ ID NO: 4 - DNA, 21 bp, Artificial Sequence
  Oligonucleotide probe
ggcacagatttcttgaagagc

SEQ ID NO: 5 - DNA, 22 bp, Artificial Sequence
  Oligonucleotide probe
gactcagaggagagagataagg

SEQ ID NO: 6 - DNA, 21 bp, Artificial Sequence
  Oligonucleotide probe
gcaaaccagaagtccttaggg

(19) United States
(12) Patent Application Publication
(10) Pub. No.: US 2011/0107445 A1
(43) Pub. Date: May 5, 2011

(54) Efficient Insertion of DNA Into Embryonic Stem Cells

(75) Inventors: Jost Seibler, Koln (DE); Nico Scheer, Koln (DE)

(73) Assignee: ITI Scotland Limited

(21) Appl. No.: 12/934,337

(22) PCT Filed: Mar. 25, 2009
(86) PCT No.: PCT/GB2009/000790

§ 371 Date: Jan. 18, 2011

(30) Foreign Application Priority Data
Mar. 26, 2008 (GB) 0805487.6
Feb. 24, 2009 (GB) 0903120.4

(51) Int. Cl. A01K 67/027; C12N 15/85

(52) U.S. Cl. 800/18; 435/462; 800/24; 800/13

(57) ABSTRACT

The present invention relates, in general, to a method for introducing a heterologous replacement gene sequence into a host embryonic stem cell to replace an endogenous host gene target sequence. In particular, the invention relates to a method for inserting large pieces of DNA into embryonic stem cells with improved efficiency, by first deleting the endogenous host gene target sequence, and subsequently utilising two proximally positioned site-specific recombinase target (RT) sites to insert a heterologous replacement gene sequence into the host chromosome.

### FIELD OF THE INVENTION

The present invention relates, in general, to a method for introducing a heterologous replacement gene sequence into a host embryonic stem cell to replace an endogenous host gene target sequence. In particular, the invention relates to a method for inserting large pieces of DNA into embryonic stem cells with improved efficiency, by first deleting the endogenous host gene target sequence, and subsequently utilising two proximally positioned site-specific recombinase target (RT) sites to insert a heterologous replacement gene sequence into the host chromosome.

### BACKGROUND TO THE INVENTION

For many years, there has been an interest in replacing an endogenous gene sequence in a cell with a heterologous replacement gene sequence. Amongst other things, this technology is used in the production of humanised mouse models. Mouse models are an invaluable tool for investigating human disease, and are used extensively to study the progression of many diseases, to test potential therapeutics, for pre-clinical studies of drug candidates and to investigate toxicology. Conventionally, transgenic mice have been produced through pronuclear injection of exogenous DNA. More recently, mice have been generated by fusing an embryonic stem cell with a cell containing a Bacterial Artificial Chromosome (BAC) or a Yeast Artificial Chromosome (YAC) comprising the exogenous gene of interest and a selectable marker to assess integration of the exogenous DNA segment into the embryonic cell genome, as described in WO94/02602, for example. Such methods rely on integration of the BAC or YAC into the embryonic stem cell genome through the process of homologous recombination. Due to the technical demands involved in handling BACs and YACs, and the low transfection rates of ES cells when using large DNA constructs, transgenesis in this manner is time-consuming, inefficient and inaccurate.

US2007/0061900 describes a method for the humanisation of the heavy and light chain immunoglobulin variable region gene loci. This method involves the insertion into each of two vectors, termed LTVECs, of a site-specific recombination site arranged so as to be contiguous to a portion of the human immunoglobulin variable region. These LTVECs are then linearised and introduced into the genome of a mouse cell by homologous recombination, so that the site-specific recombination sites flank the mouse immunoglobulin variable region sequences, and the partial human immunoglobulin variable region sequences flank the site-specific recombination sites. Effecting site-specific recombination excises the mouse immunoglobulin variable region sequence and joins the two partial human immunoglobulin variable region sequences, with the residual site-specific recombination site contained within it. The resulting mice produce hybrid antibodies containing human variable and mouse constant regions, with subsequent transformation steps required to allow production of pure human antibodies. However, this approach is inefficient, due to the low frequency of homologous recombination with vectors that carry very large sequences of heterologous DNA. Furthermore, the segmental nature of the immunoglobulin variable region allows the residual site-specific recombination site to remain within the nucleic acid sequence with little chance of a detrimental effect. There is no indication that technology of this type might be utilised for the humanisation of non-segmental genes, where the presence of a nucleic acid sequence coding for a residual site-specific recombination site within the gene might compromise its ability to be transcribed.

Wallace et al. (Cell 128, 197-209 2007) recently described a method known as recombinase-mediated genomic replacement (RMGR) which is a more generally applicable system for exchanging an endogenous gene sequence with a heterologous replacement. This method also utilises site-specific recombination to replace a mouse allele with the human allele of the orthologous gene. Two non-interacting site-specific recombination sites (loxP and lox511) are inserted into the mouse chromosome flanking the target gene by homologous recombination. An identical pair of non-interacting site specific recombination sites are inserted into a BAC so as to flank the human allele of the target gene. The introduction of the BAC into the mouse cell, and the subsequent expression of the site-specific recombinase result in two site-specific recombination reactions between the compatible site-specific recombination sites of the BAC and the mouse chromosome (loxP/loxP and lox511/lox511), and the reciprocal exchange of the human gene sequence for the mouse sequence.

However, this method is inefficient for the insertion of large pieces of DNA due to the considerable distance between the non-interacting site-specific recombination sites present on the mouse chromosome and in the BAC. This distance is inevitable due to the presence of the mouse allele in the mouse chromosome at the time of recombination, and the reciprocal nature of the recombinatorial exchange. Furthermore, a closer analysis of these clones demonstrates that some of them show a rearrangement in the BAC DNA, resulting in an even lower frequency of correctly targeted clones. Besides this, Wallace et al. use the reconstitution of a functional hypoxanthine-phosphoribosyltransferase (Hprt) minigene from 5′ to 3′ components in order to select for correctly targeted clones. Therefore, this approach is only possible in an HPRT-deficient (hprt⁻) embryonic stem cell line.

There thus remains a need for a more efficient method for replacing an endogenous gene sequence in a cell with a heterologous replacement gene sequence, which would overcome some of these problems of inefficiency.

In addition, the invention sets out to provide more generally applicable methods for selecting correctly targeted clones. Provision of an improved general method of selecting such clones will allow recombinatorial exchange to be performed in cells other than an HPRT-deficient (hprt⁻) embryonic stem cell line. A universal selection method would also allow such a procedure to be conducted in any embryonic stem cell.

### SUMMARY OF THE INVENTION

According to the present invention, there is provided a method of introducing a heterologous replacement gene sequence into a host cell to replace an endogenous host gene target sequence, the method comprising:

a) incorporating a pair of identical site-specific recombinase target (RT) sites of type I into the same allele of a host chromosome in separate homologous recombination steps such that the endogenous host gene target sequence that is to be replaced is flanked on each side by said identical type I RT sites; wherein one of the identical type I RT sites is flanked by a type II RT site positioned proximal to the type I RT site, wherein the type II RT site is different to the type I RT site such that it is heterospecific, and as such cannot interact with the type I RT site and;
b) effecting recombination between said pair of type I site-specific recombination sites such that the endogenous host gene target sequence is excised, and whereby a residual type I RT site remains in the chromosome at the excision point; and
c) bringing a heterologous replacement gene sequence into contact with the host chromosome, whereby the heterologous replacement gene sequence is flanked on one side by a type I RT site and on the other side by a type II RT site, under appropriate conditions to effect targeted site-specific recombinase mediated insertion of the heterologous replacement gene sequence into the host chromosome by effecting recombination between corresponding type I and type II site-specific recombination sites flanking the heterologous replacement gene sequence and located in the host chromosome, such that the heterologous gene sequence is introduced at the position in the host chromosome previously occupied by the host target gene.

A simple schematic of the mechanism of the invention is shown in FIG. 1. In brief, two type 1 RT sites are incorporated into the endogenous host cell chromosome of a host cell by two separate conventional homologous recombination reactions. Homologous recombination is a phenomenon well known in the art, yet for ease of comprehension, a schematic of the mechanism of homologous recombination is shown in FIG. 2. The two recombination reactions are facilitated by short regions of homology between the endogenous host cell chromosome and the replacement nucleic acid sequence that comprises the type I RT site. These regions of homology facilitate strand invasion and subsequent base pairing, allowing strand elongation which inserts each recombination site into the host cell chromosome.

In addition to the type I RT sites, which are inserted on either side of the endogenous host gene target sequence, on one side of the endogenous host gene target sequence a type II RT site is also incorporated into the endogenous host cell chromosome, proximal to, and flanking the type I RT site. The insertion of the two type I RT sites and one type II RT site into the endogenous host cell chromosome results in the arrangement shown in FIG. 1. Once both recombination reactions are complete, the type I RT sites should thus flank the endogenous host gene target sequence, with one of the type I RT sites additionally flanked by a type II RT site, such that the type I RT site is positioned between the type II RT site and the endogenous host gene target sequence. Both type I RT sites should be aligned in the same direction, as shown in FIG. 1, so as to allow their recombination together in due course. It is known in the art that site-specific recombinases can be utilised to target homologous recombination to specific chromosomal locations (see Jessen et al., 1997). The use of such site specific recombinases allows recombination to be initiated upon demand by the addition of the site-specific recombinase.

In order to excise the endogenous host gene target sequence, site-specific recombination can then be effected between the two type I RT sites in the host cell. For ease of comprehension, the mechanism of site specific recombination is illustrated in FIG. 3. These recombination events result in excision of the endogenous host gene target sequence, leaving the residual type I RT site positioned proximal to the type II RT site within the host cell chromosome. This intermediate stage represents the production of a host cell in which the endogenous gene is knock-out (a knock-out ES cell).

The next stage in the methodology is to provide a heterologous replacement gene sequence. The heterologous replacement gene sequence is located between a flanking type I RT site and a flanking type II RT site. The RT sites are aligned in the same direction as the corresponding RT sites in the host cell chromosome, so as to allow their recombination together in due course. The heterologous replacement gene sequence may be located within a vector, or may be a linear nucleic acid sequence. Preferably, the heterologous replacement gene sequence is located within a vector.

In order to insert the heterologous replacement gene sequence into the host cell chromosome, site-specific recombination is then effected between the corresponding RT sites present on the host cell chromosome and flanking the heterologous replacement gene sequence. The mechanism of recombination is as depicted in FIG. 3. These recombination events result in the insertion of the heterologous replacement gene sequence into the host cell chromosome, at the position in the host chromosome previously occupied by the endogenous host gene target sequence, and flanked by the residual type I RT site on one side and the residual type II RT site on the other.

The method of the invention has a number of advantages. Firstly, the use of site-specific recombination for the insertion of the heterologous replacement gene sequence into the host cell chromosome allows for greatly improved efficiency over homologous recombination. The method described in US2007/0061900 utilises homologous recombination for the insertion of a portion of the human immunoglobulin variable region which is contained within a linearised LTVEC. The large size of the replacement sequence necessitates long DNA homology arms to facilitate homologous recombination between the host cell chromosome and the heterologous replacement gene sequence, which leads to corresponding inefficiencies. In contrast, the method of the present invention utilises site-specific recombination, which does not require long DNA homology arms to effect recombination, and the efficiency is therefore greatly improved. In addition, the use of site-specific recombination for the insertion of the heterologous replacement gene sequence negates the need for large size homology arms, and allows the verification of correctly targeted cell clones by Southern blot analysis.

When the mechanism of site-specific recombination is used for the insertion of the heterologous DNA sequence, as in the present invention, a greatly improved efficiency is evident. The method of the present invention allows the complete replacement of an endogenous host gene target sequence with a heterologous replacement gene sequence in just a few rounds of targeting in host cells.

Additionally, excision of the endogenous host gene target sequence generates a knock-out cell, which acts as an intermediate in the method. This can be usefully exploited, separately from the ultimate goal of successful introduction of the heterologous gene sequence, and allow analysis of the function of the excised endogenous host gene target sequence by looking at the effect of its deletion. The ultimate insertion of the heterologous replacement gene sequence can allow comparison of the function of the replacement gene sequence with that of the endogenous gene sequence and the complete knock-out.

A further advantage of the present invention concerns the proximal positioning of the type I and type II RT sites in the host cell chromosome after excision of the endogenous gene sequence. As described in Wallace, the frequency of correct targeting in a host chromosome where the RT sites are separated on different entities is less than 1×10⁻⁸. According to the method of the present invention, following excision of the endogenous host gene target sequence, the residual type I RT sequence and the type II RT sequence are positioned proximally Preferably, a “proximal” position resides within 100 nucleotides, preferably within 50 nucleotides, more preferably, within 40, 30, 20, 15, 10, 5 or less of another. This positioning greatly increases the efficiency of insertion of the heterologous replacement gene sequence, and has lead to a targeting efficiency with the method of the present invention which can be as high as 1×10⁻⁶. This is an important benefit, as obtaining correctly targeted embryonic stem cells is generally the rate limiting step in the generation of embryonic stem cells with a replacement of an endogenous host gene target sequence with a heterologous replacement gene sequence.

Furthermore, because the lengths of DNA used by Wallace are so long (of the order of 200 kb), there is a much increased opportunity for intramolecular rearrangements and undesired homologous recombination events to occur, which increases the chance of a non-functional or incorrect DNA structure being created. The method of the present invention is advantageous in view of the Wallace method because the greatly increased efficiency allows the skilled person to start with many more clones in order to identify those in which the integrity and fidelity of the heterologous sequence is maintained.

The introduced heterologous replacement gene sequence may be incorporated under the control of its own regulatory sequences. Alternatively, the genetic recombination events can be arranged so that the equivalent host cell regulatory sequences are situated upstream of the inserted heterologous replacement gene sequence and thus used in their place. Sometimes it will be desired to retain the host cell regulatory sequences rather than incorporate the regulatory sequences that are thought to govern transcription of the heterologous replacement gene sequence. For example, in some cases the regulatory sequences associated with the heterologous replacement gene sequence may be unable to control expression of the heterologous replacement gene sequence in the host cell. This is shown by Cheung et al (Journal of Pharmacology and Experimental Therapeutics 316, 1328-1334 2006), where it is demonstrated that in a mouse humanised for CYP3A4 which carries the human promoter for CYP3A4 protein is not expressed in adult males. It is thus supposed that some promoter element or other factor must exist that is missing from the construct used. In contrast, by retaining the mouse regulatory sequences and using these for regulation instead of the human sequences, one can guarantee that faithful regulation of the introduced gene will be retained and such problems avoided.

One advantage of the methodology of the invention over conventional techniques, where integration of a heterologous replacement gene sequence into a host cell chromosome is more or less random, is that integration at the site of the equivalent host cell gene sequence ensures that the genomic context of gene placement is retained. By integrating at such a site, it is likely that the local chromosome structure is “open” in the sense that access to the chromosomal DNA is possible for transcription factors and other proteins required for transcription to take place. Not only this, but the same chromosomal context is retained as for the endogenous host gene sequence, such that regulation of DNA transcription at the level of the tertiary structure of the chromosome, by way of histone binding, and local folding/unfolding of the chromosome, is retained. This ensures that holistic regulation of gene transcription is retained, such that the same tissue distribution of gene regulation is followed for the introduced heterologous replacement gene sequence as that seen for the endogenous host gene sequence. This complete retention of physiological regulation mechanisms at the gene transcription level is not common to prior art techniques.

### BRIEF DESCRIPTION OF THE FIGURES

FIG. 1. Schematic of the methodology of the invention. The method provides a mechanism of introducing a heterologous replacement gene sequence into a host embryonic stem cell to replace an endogenous host gene target sequence, comprising the insertion of two type I RT sites to flank the endogenous host gene target sequence, one of which is flanked by a type II RT sequence, effecting site-specific recombination between the type I RT sites to excise the endogenous host gene target sequence, providing a vector comprising a heterologous replacement gene sequence flanked by a type I RT site and a type II RT site, and effecting recombination between the corresponding RT sites present on the host cell chromosome and on the vector such that the heterologous gene sequence is introduced at the position in the host chromosome previously occupied by the host target gene.

FIG. 2. Mechanism of homologous recombination. Homologous recombination occurs following a double stranded chromosomal break. 5′ to 3′ exonuclease activity produces a 3′ overhang and allows strand invasion to occur. DNA synthesis utilises the intact strand as a template and ligation repairs the chromosomal break generating a Holliday junction. Subsequent branch migration and resolution produce recombinant products.

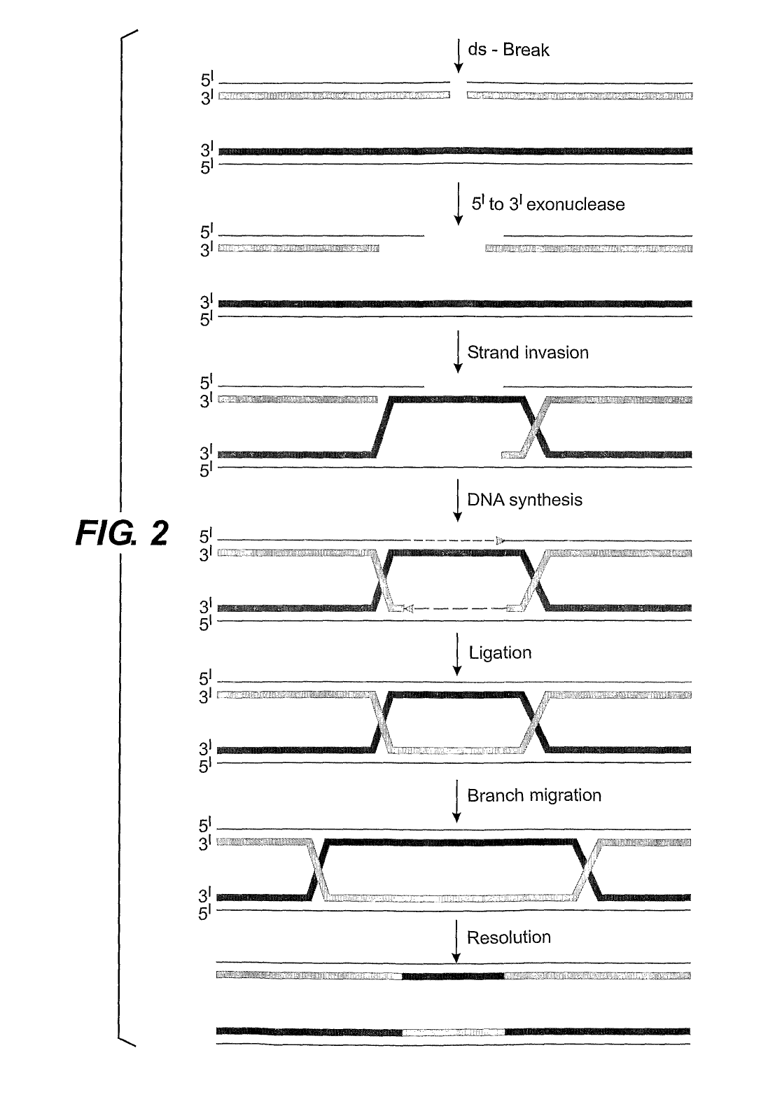

FIG. 3. A) The LoxP site-specific recombination site. B) Mechanism of site-specific recombination. Two LoxP sites align through complementary base pairing, allowing Cre recombinase to catalyse recombination between the 2 sites, so excising the endogenous host gene target sequence.

FIG. 4. Method for the production of a transgenic mouse. A transgenic mouse is produced by the insertion of one or more altered embryonic stem cells into a developing blastocyst. The blastocyst is then implanted into a pseudo-pregnant mouse and allowed to develop, producing a chimera.

FIG. 5. Strategy for the deletion of the mouse Cyp3a cluster. (A) Schematic representation of the chromosomal organisation and orientation of functional genes within the mouse Cyp3a Cluster (adapted from Nelson et al., 2004). Pseudogenes are not listed. (B) Exon/Intron structure of Cyp3a57 and Cyp3a59. Exons are represented as black bars and the ATGs mark the translational start sites of both genes. The positions of the targeting arms for homologous recombination are highlighted as light (Cyp3a57) and dark (Cyp3a59) grey lines, respectively. (C) Vectors used for targeting of Cyp3a57 (left) and Cyp3a59 (right) by homologous recombination. LoxP, lox5171, frt and f3 sites are represented as white, striped, black or grey triangles, respectively. (D) Genomic organisation of the Cyp3a Cluster in double targeted ES cells after homologous recombination on the same allele at the Cyp3a57 and Cyp3a59 locus. (E) Deletion of the mouse Cyp3a Cluster after Cre-mediated recombination at the loxP sites. All exons and introns from Cyp3a57, Cyp3a16, Cyp3a41, Cyp3a44, Cyp3a11 and Cyp3a25 are completely deleted and Exons 1 to 4 and the promoter of Cyp3a59. Therefore, the only functional Cyp3a gene that remains after Cre-mediated deletion is Cyp3a13, which is separated from the rest of the Cluster by >7 Megabases (Mb) genomic DNA and a number of functional Cyp-unrelated genes. Primers used to demonstrate successful deletion of the mouse Cyp3a Cluster are depicted as black arrows.

For the sake of clarity sequences are not drawn to scale. TK=Thymidine Kinase expression cassette, Hygro=Hygromycine expression cassette, ZsGreen=ZsGreen expression cassette, P=Promoter that drives the expression of Neomycin, 5′Δ Neo=ATG-deficient Neomycin.

FIG. 6: Strategy for the humanisation of the mouse Cyp3a Cluster. (A) Initial configuration after Cre-mediated deletion of the Cyp3a Cluster as already depicted in FIG. 5E. (B) Modified human BAC comprising the human CYP3A4 and CYP3A7 genes used for Cre-mediated insertion into the deleted mouse Cyp3a Cluster. (C) Genomic organisation of the Cyp3a Cluster in correctly targeted ES cells after Cre-mediated insertion of the human BAC. (D) Deletion of the hygromycin and neomycin selection cassettes after Flp-mediated recombination at the frt and f3 sites.

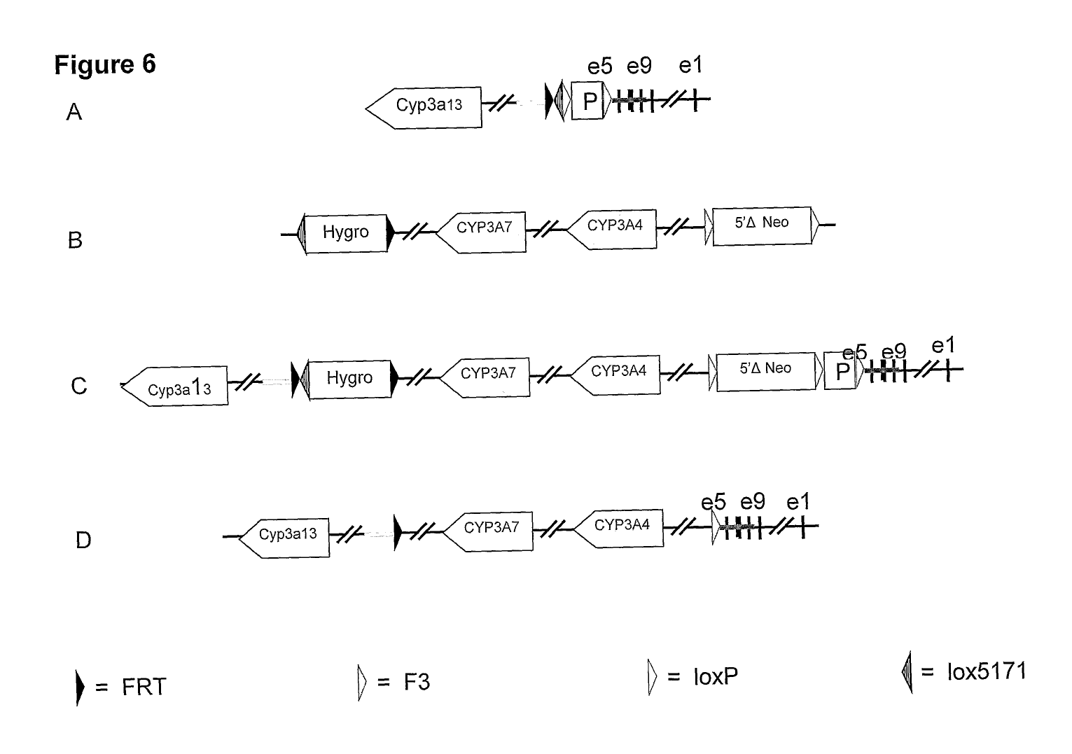

For the sake of clarity sequences are not drawn to scale. Hygro=Hygromycine expression cassette, P=Promoter that drives the expression of Neomycin, 5′Δ Neo=ATG-deficient Neomycin.

FIG. 7. PCR analysis of 3 G418 resistant clones (A) Genomic organisation of the Cyp3a gene cluster in correctly targeted ES cells after Cre-mediated insertion of the human BAC, as depicted in FIG. 6C. PCR primers used for PCR analysis are shown as black arrows, and expected PCR fragments are show as grey boxes. (B) PCR results showing that all 3 clones carry a correct insertion of the human BAC.

FIG. 8. Southern analysis of 3 G418 resistant clones (A) Genomic organisation of the Cyp3a gene cluster in correctly targeted ES cells after Cre-mediated insertion of the human BAC, as depicted in FIG. 6C. The Southern probe used for Southern blot analysis is shown as a black line, and the expected restriction fragments are indicated. (B) Southern blot results showing that all clones carry a correct insertion of the human BAC, and that clone 3 has an additional insertion.

FIG. 9. Hepatic CYP3A4 protein in humanised CYP3A4 mouse lines Southern blot results showing the presence of human CYP3A4 in the liver of Cyp3a knockout mice.

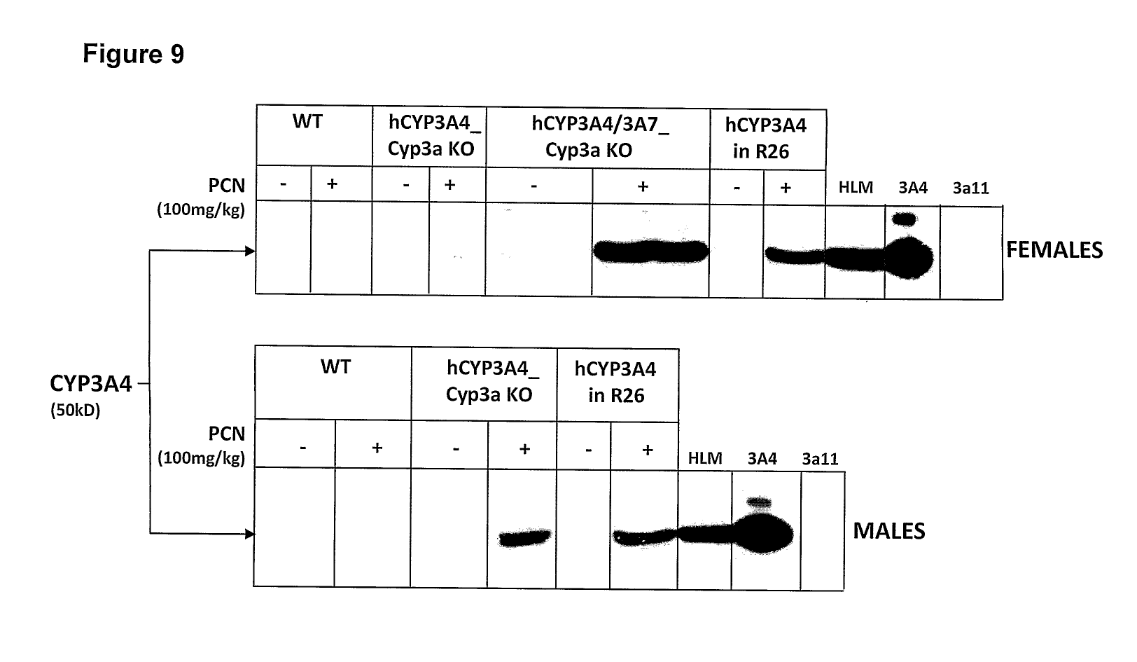

FIG. 10. Intestinal CYP3A4 protein in humanised CYP3A4 mouse lines Southern blot results showing the presence of human CYP3A4 in the intestine of Cyp3a knockout mice.

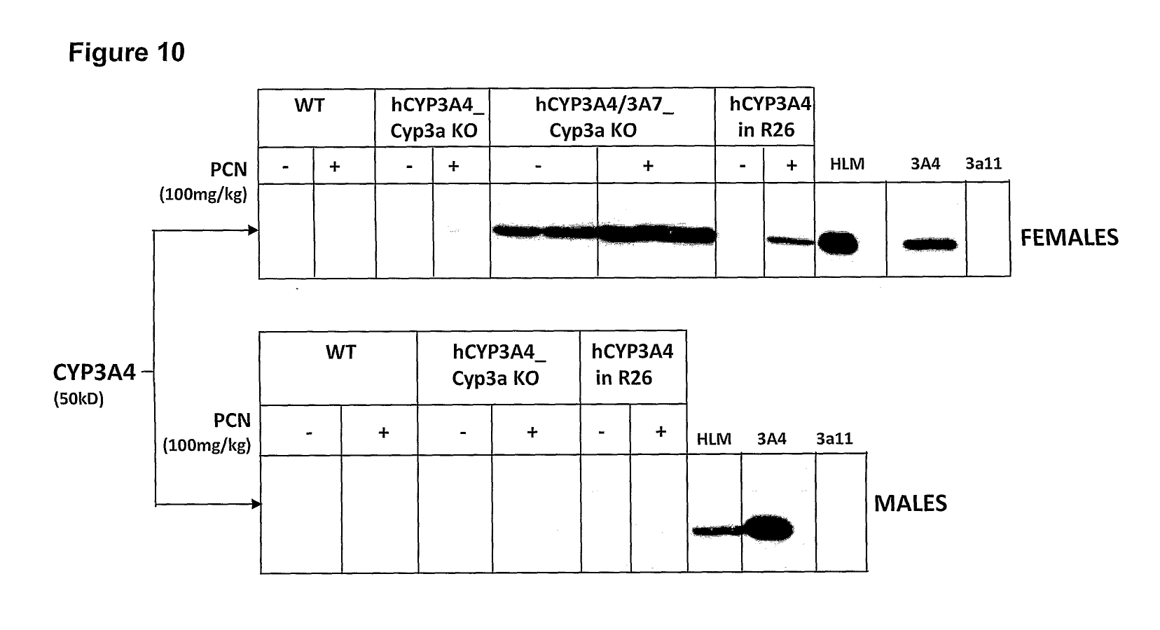

FIG. 11. DNA analysis of CYP3A4/3A7 humanised mice Sequence alignments with the CYP3A4 cDNA in the final construct showed that the CYP3A4 cDNAs cloned from humanised mice lines (hCYP3A4/3A7_Cyp3a KO and hCYP3A4_Cyp3a KO) were full-length transcripts, and there was no mutation in the sequences. No CYP3A7 transcripts detected in hCYP3A4/3A7_Cyp3a KO.

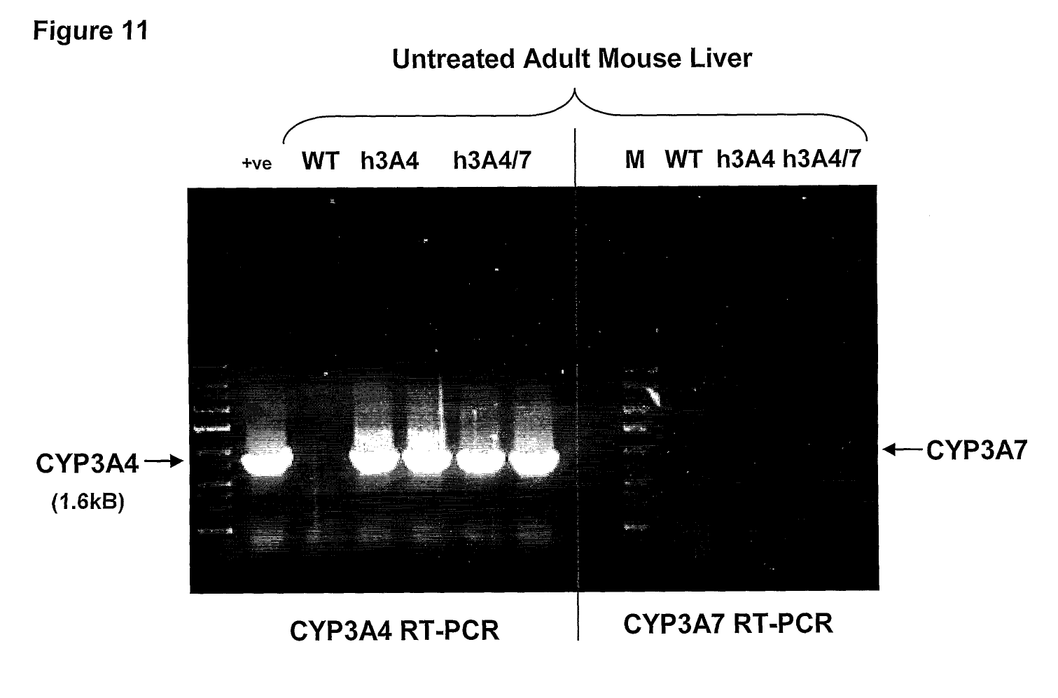

FIG. 12. Dehydroepiandrosterone metabolism in fetal, paediatric and adult humans CYP3A7 is the major CYP3A isoform expressed in human fetal liver, undergoes a developmental switch in the first week of postnatal life, with CYP3A7 virtually disappearing concomitant with transcriptional activation of the CYP3A4 gene.

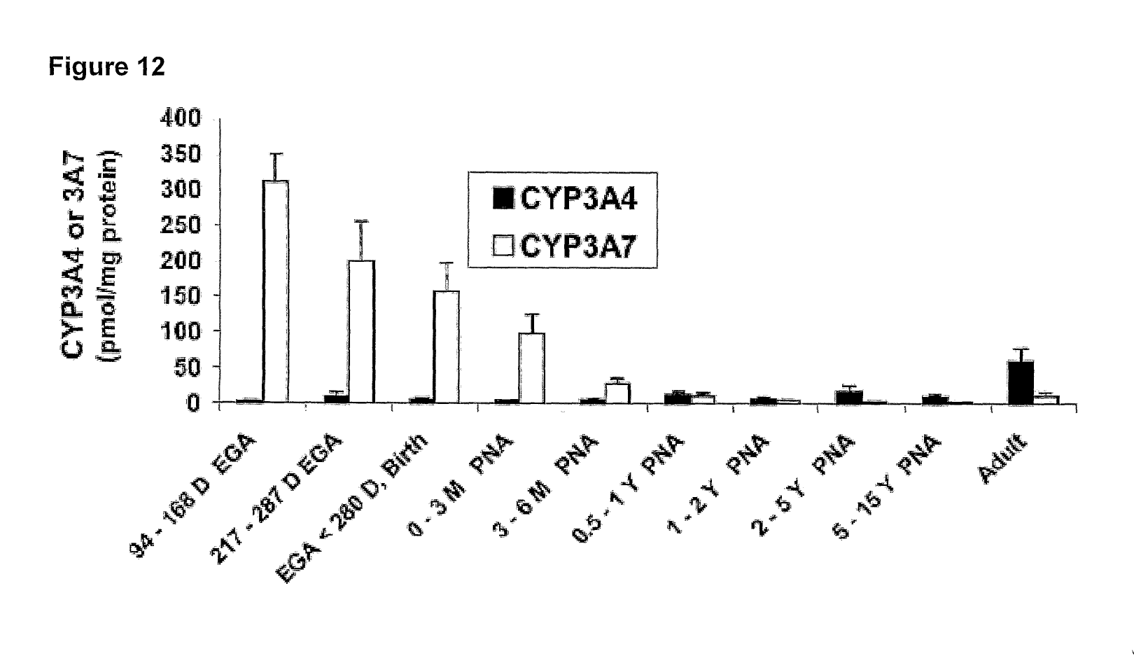

A similar developmental switch has also been observed in the mouse (Cyp3a16 to Cyp3a11). The mouse used our experiment was over 9 weeks old and therefore, the expression of CYP3A7 might be switched to CYP3A4.

FIG. 13. Hepatic CYP3A4 and Cyp3a protein expression in humanised CYP3A4 mouse lines Southern blot results showing the presence of human CYP3A4 in the liver of Cyp3a knockout mice.

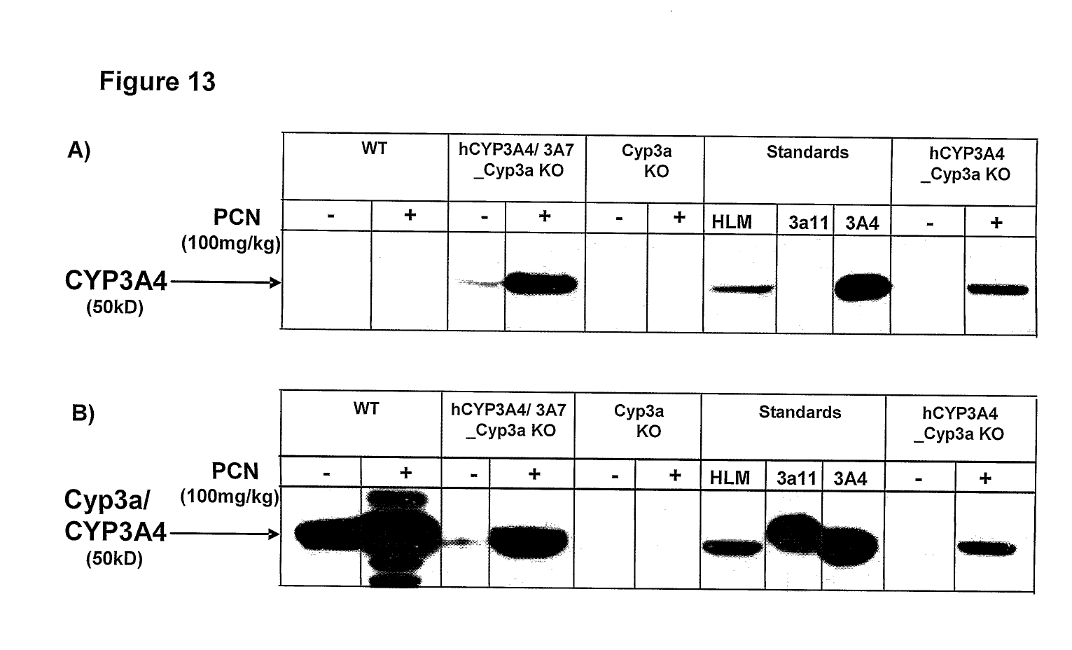

FIG. 14. Intestinal CYP3A4 and Cyp3a protein expression in humanised CYP3A4 mouse lines Southern blot results showing the presence of human CYP3A4 in the intestine of Cyp3a knockout mice.

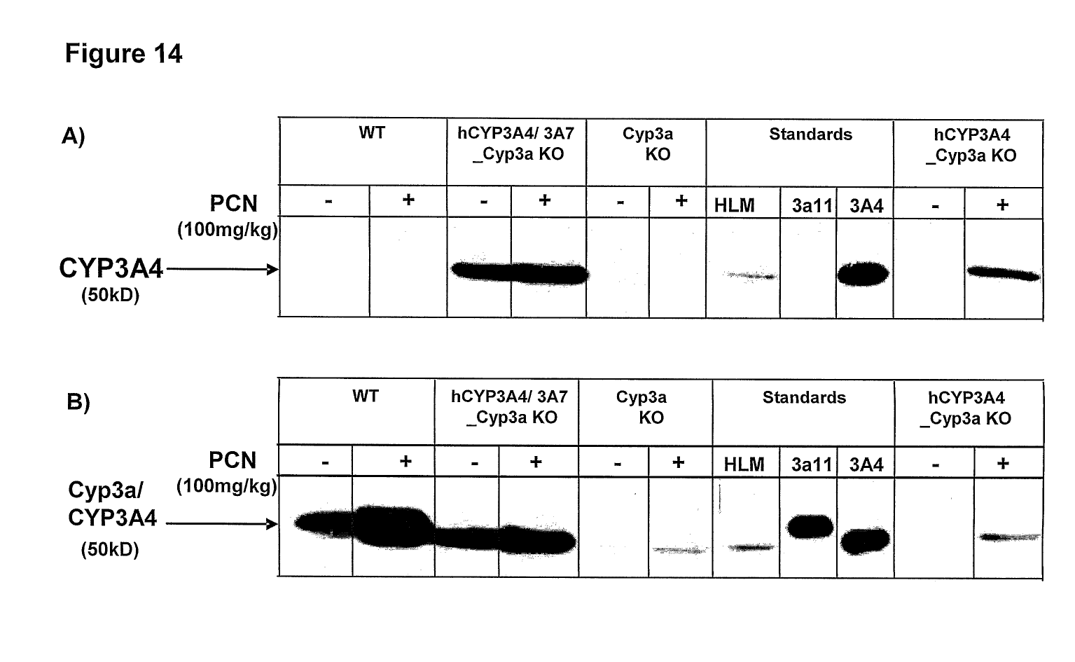

FIG. 15. CYP3A4 is catalytically active in CYP3A4/3A7_Cyp3a KO mice, as shown by Triazolam Oxidation Relative to Cyp3a KO mice, there is increased TRI metabolism due to the high catalytic activity of CYP3A4 in hCYP3A4/3A7_Cyp3a KO mice. CYP3A4 plays a significant role in TRI metabolism in the liver, however TRI can also be extensively metabolised in the mouse.

FIG. 16. CYP3A4 is catalytically active in CYP3A4/3A7_Cyp3a KO mice, as shown by Triazolam Oxidation A. Triazolam oxidation results showing catalytic activity of CYP3A4 in the liver of CYP3A4/3A7 Cyp3a knockout mice B. Triazolam oxidation results showing catalytic activity of CYP3A4 in the duodenum of CYP3A4/3A7 Cyp3a knockout mice.

FIG. 17. CYP3A4 is catalytically active in CYP3A4/3A7_Cyp3a KO mice, as shown by DBF Oxidation A. DBF oxidation results showing catalytic activity of CYP3A4 in the liver of CYP3A4/3A7 Cyp3a knockout mice B. DBF oxidation results showing catalytic activity of CYP3A4 in the duodenum of CYP3A4/3A7 Cyp3a knockout mice.

FIG. 18. CYP3A4 is catalytically active in CYP3A4/3A7_Cyp3a KO mice, as shown by BQ Oxidation A. BQ oxidation results showing catalytic activity of CYP3A4 in the liver of CYP3A4/3A7 Cyp3a knockout mice B. BQ oxidation results showing catalytic activity of CYP3A4 in the duodenum of CYP3A4/3A7 Cyp3a knockout mice.

FIG. 19. Clinical chemistry analysis of plasma from C57BL/6J, hCYP3A4/3A7_Cyp3a KO, hCYP3A4_Cyp3a KO and Cyp3a KO mice: (A) triglycerides (B) low density lipoproteins (LDL) (C) high density lipoprotein (HDL) (D) cholesterol (CHOL). Data shown are mean±S.D. (n=3 for C57BL/6J mice, n=2 for all PCN treated transgenic animals). Data from the treated groups were compared with an unpaired t test (two tailed P values); *—Significantly different compared to the treated C57BL/6J mice (*—P<0.05; **—P<0.01).

FIG. 20. Clinical chemistry analysis of plasma from C57BL/6J, hCYP3A4/3A7_Cyp3a KO, hCYP3A4_Cyp3a KO and Cyp3a KO mice (A) total bilirubin (BIL-T) (B) direct bilirubin (BIL-D) (C) aspartate aminotransferase (AST) (D) alanine aminotransferase (ALT). Data shown are mean±S.D. (n=3 for C57BL/6J mice, n=2 for all PCN treated transgenic animals). Data from the treated groups were compared with an unpaired t test (two tailed P values); *—Significantly different compared to the treated C57BL/6J mice (*—P<0.05).

FIG. 21. Clinical chemistry analysis of plasma from C57BL/6J, hCYP3A4/3A7_Cyp3a KO, hCYP3A4_Cyp3a KO and Cyp3a KO mice (A) alkaline phosphatase (ALP) (B) albumin (ALB). Data shown are mean±S.D. (n=3 for C57BL/6J mice, n=2 for all PCN treated transgenic animals). Data from the treated groups were compared with an unpaired t test (two tailed P values); *—Significantly different compared to the treated C57BL/6J mice (***—P<0.001).

FIG. 22. CYP3A4 protein expression in (A) liver and (B) intestinal microsomes from C57BL/6J, hCYP3A4/3A7_Cyp3a KO, hCYP3A4_Cyp3a KO and Cyp3a KO mice (+)—treated with PCN (100 mg/kg/2 days/IP); (−)—control animals treated with vehicle (corn oil). Each lane is a sample from one animal. 10 μg of liver or 20 μg of intestinal microsomal protein were loaded. Blots were incubated in a polyclonal rabbit anti-CYP3A4 (Gentest, cat #458234). Standards: HLM—pooled male human liver microsomes (10 μg) (Gentest, cat #452172); 3a11—murine Cyp3a11 recombinant protein (0.1 pmol) (Dr. Henderson, Uni. of Dundee, UK); 3A4—human CYP3A4 baculosomes (0.1 pmol) (Invitrogen, cat #P2377).

FIG. 23. CYP3A/Cyp3a protein expression in (A) liver and (B) intestinal microsomes from C57BL/6J, hCYP3A4_Cyp3a KO, hCYP3A4_Cyp3a KO and Cyp3a KO mice (+)—treated with PCN (100 mg/kg/2 days/IP); (−)—control animals treated with vehicle (corn oil). Each lane is a sample from one animal. 10 μg of liver or 20 μg of intestinal microsomal protein were loaded. Blots were incubated in a polyclonal rabbit anti-rat CYP3A2 (Dr. Henderson, Uni. of Dundee, UK). Standards: HLM—pooled male human liver microsomes (10 μg) (Gentest, cat #452172); 3a11—mouse Cyp3a11 recombinant protein (0.1 pmol) (Dr. Henderson, Uni. of Dundee, UK); 3A4-human CYP3A4 baculosomes (0.1 pmol) (Invitrogen, cat #P2377). The control band for Cyp3a11 demonstrated less than 50 kD electrophoretic mobility and this is attributed to the fact that the protein was histidine tagged.

FIG. 24. 7-BQ oxidation by liver (A) and intestinal (B) microsomes from C57BL/6J, hCYP3A4/3A7_Cyp3a KO, hCYP3A4_Cyp3a KO and Cyp3a KO mice Data generated according to CXR approved Laboratory Method Sheet Fluor-0005. Apart from bars for untreated transgenic/knock-out mice, which represent a single measurement, data are mean±SD (n=3 for C57BL/6J microsomes; n=2 for microsomes from PCN treated transgenic strains and human liver microsomes (HLM)). Activities of samples from treated hCYP3A4/3A7_Cyp3a KO and hCYP3A4_Cyp3a KO mice were compared to that from Cyp3a KO line with an unpaired t test (two tailed P values).

FIG. 25. DBF oxidation by liver (A) and intestinal (B) microsomes from C57BL/6J, hCYP3A4/3A7_Cyp3a KO, hCYP3A4_Cyp3a KO and Cyp3a KO mice. Apart from bars for untreated transgenic/knock-out mice and human liver microsomes (HLM), which represent a single measurement, data are mean±SD (n=3 for C57BL/6J microsomes; n=2 for microsomes from PCN treated transgenic strains). Activities of samples from treated hCYP3A4/3A7_Cyp3a KO and hCYP3A4_Cyp3a KO mice were compared to that from Cyp3a KO line with an unpaired t test (two tailed P values). *—Significantly different (*—P<0.05; **—P<0.01; ***—P<0.001).

FIG. 26. α-Hydroxylation of triazolam by liver (A) and intestinal (B) microsomes from C57BL/6J, hCYP3A4/3A7_Cyp3a KO, hCYP3A4_Cyp3a KO and Cyp3a KO mice In part A activities of samples from vehicle treated mice should be read using left Y axis scale, whereas activities of microsomes from PCN treated animals should be read using right Y axis scale. Apart from bars for untreated transgenic/knock-out mice, which represent a single measurement, data are mean±SD (n=3 for C57BL/6J microsomes; n=2 for microsomes from PCN treated transgenic strains and human liver microsomes (HLM)). Activities of samples from treated hCYP3A4/3A7_Cyp3a KO and hCYP3A4_Cyp3a KO mice were compared to that from Cyp3a KO line with an unpaired t test (two tailed P values). *—Significantly different (*—P<0.05; **—P<0.01)

FIG. 27. Agarose gel electrophoresis of RT-PCR products The reactions used CYP3A4 (lines 1-5) and CYP3A7 (lines 7-9) specific primers and total liver RNA. (1)—C57BL/6J; (2-3)—hCYP3A4/3A7_Cyp3a KO; (4-5)—hCYP3A4_Cyp3a KO; (6)—molecular weight marker 1 kb ladder, (7)—C57BL/6J; (8-9)—hCYP3A4/3A7_Cyp3a KO.

### DETAILED DESCRIPTION OF PREFERRED EMBODIMENTS

The invention provides a method of introducing a heterologous replacement gene sequence into a host cell to replace an endogenous host gene target sequence, the method comprising:

a) incorporating a pair of identical site-specific recombinase target (RT) sites of type I into the same allele of a host chromosome in separate homologous recombination steps such that the endogenous host gene target sequence that is to be replaced is flanked on each side by said identical type I RT sites; wherein one of the identical type I RT sites is flanked by a type II RT site positioned proximal to the type I RT site, wherein the type II RT site is different to the type I RT site such that it is heterospecific, and as such cannot interact with the type I RT site and;

b) effecting recombination between said pair of type I site-specific recombination sites such that the endogenous host gene target sequence is excised, and whereby a residual type I RT site remains in the chromosome at the excision point; and

c) bringing a heterologous replacement gene sequence into contact with the host chromosome, whereby the heterologous replacement gene sequence is flanked on one side by a type I RT site and on the other side by a type II RT site, under appropriate conditions to effect targeted site-specific recombinase mediated insertion of the heterologous replacement gene sequence into the host chromosome by effecting recombination between corresponding type I and type II site-specific recombination sites flanking the heterologous replacement gene sequence and located in the host chromosome, such that the heterologous gene sequence is introduced at the position in the host chromosome previously occupied by the host target gene.

According to the present invention, a heterologous replacement gene sequence is inserted into the chromosome of the host cell at the point in the chromosome where the endogenous host gene target sequence naturally occurs. This has the advantage that the context of the gene locus is retained which means that the fidelity of transcription from this site is as close as possible to the level of transcription that occurs in the wild type system.

### Methodology

The first stage of the method of the present invention is the incorporation of a pair of identical type I RT sites into the host cell chromosome. Methods for incorporation of the RT sites into the chromosome will be known to those of skill in the art, and are preferably performed by exploiting the process of homologous recombination. Homologous recombination relates to the genetic mechanism which can be exploited to allow the insertion of a nucleic acid sequence into the host cell chromosome. The mechanism is initiated by the alignment of double-stranded host cell and exogenous nucleic acid sequences. A double strand break in the host cell sequence and 5′ to 3′ exonuclease activity facilitates strand invasion, resulting in pairing of the homologous host cell and exogenous sequences through short regions of homology. Subsequent chain elongation of the host cell sequence utilises the exogenous sequence as a template and resolution produces the host cell genomic sequence with the exogenous sequence located within it, whilst the exogenous sequence remains intact.

Methods for performing homologous recombination are known in the art and exploit regions of homology between exogenously supplied DNA molecules and the target chromosome to introduce the RT sites. Examples of suitable targeted delivery systems will be clear to those of skill in the art and include the use of injected or targeted naked DNA, targeted liposomes encapsulating and/or complexed with the DNA, targeted retroviral systems and targeted condensed DNA such as protamine and polylysine-condensed DNA, or electroporation. Other delivery methods may also be employed, such as by using nucleic acid expression vectors, polycationic condensed DNA or ligand linked DNA (see Curiel (1992) Hum Gene Ther 3:147-154; Wu (1989) J Biol Chem 264:16985-16987), and use of a gene transfer particle gun, (described in U.S. Pat. No. 5,149,655). Naked DNA may also be employed, as is described in detail in international patent application WO90/11092. This list is provided by way of illustration only, and is not intended to be limiting.

The recombination steps are performed in a host cell, according to methods well known in the art and discussed further below. Preferably the host cell is a stem cell, such as an iPS cell or an embryonic stem cell. Embryonic stem (ES) cells are cultured cell lines of totipotent cells, wherein the cells, when introduced into an early embryo, will develop to populate all tissues of the developing organism. ES cells are preferred host cells according to the invention.

Within the method of the present invention, each of the type I RT sites is preferably incorporated into the host cell chromosome through a separate homologous recombination step, as described above. Each of the separate homologous recombination reactions begins with the host cell chromosome and exogenous DNA which comprises the type I RT site, and regions of homology to the host cell chromosome region where homologous recombination is to occur. Preferably the regions of homology are between 1 and 6 kb, more preferably the regions of homology are between 1 and 4 kb, most preferably, one of the regions of homology is 1 kb in length, and the other is either 3 kb or 4 kb in length.

Within the method of the present invention, the two type I RT sites are incorporated into the host cell chromosome so that the endogenous host gene target sequence which is to be replaced by the heterologous replacement gene sequence is flanked on each side by a type I RT site Preferably, the two type 1 RT sites are inserted into the endogenous host gene target sequences so that they are located less than 5 mb from the host gene target sequence, more preferably the two type 1 RT sites are inserted into the endogenous host gene target sequence so that they are located less than 3 mb from the host gene target sequence, and most preferably the two type 1 RT sites are inserted into the endogenous host gene target sequence so that they are located less than 2 mb from the host gene target sequence. Further within the method of the present invention, the two type I RT sites are positioned in the same orientation as each other, to allow recombination between them in due course.

Within the method of the present invention, one of the type I RT sites incorporated into the host cell chromosome is flanked by a type II RT site, such that the type I RT site is positioned between the endogenous host gene target sequence and the type II RT site. The type II RT site is preferably incorporated into the host cell chromosome through the same recombination step as its proximal type I RT site. For this, the exogenous DNA sequence utilised in the homologous recombination step should preferably contain the DNA sequence for the type I RT site and the type II RT site so that these can be introduced together.

Within the method of the present invention, the type I RT site and the type II RT site are positioned proximal to one another. By “proximal”, as the term is used herein, is meant that the RT sites are positioned next to one another, close in proximity on the chromosome. Preferably, a “proximal” position resides within 100 nucleotides, preferably within 50 nucleotides, more preferably, within 40, 30, 20, 15, 10, 5 or less of another. As described in more detail below, the type I RT site is different from the type II RT site, such that it is heterospecific, and as such cannot interact with the type I RT site.

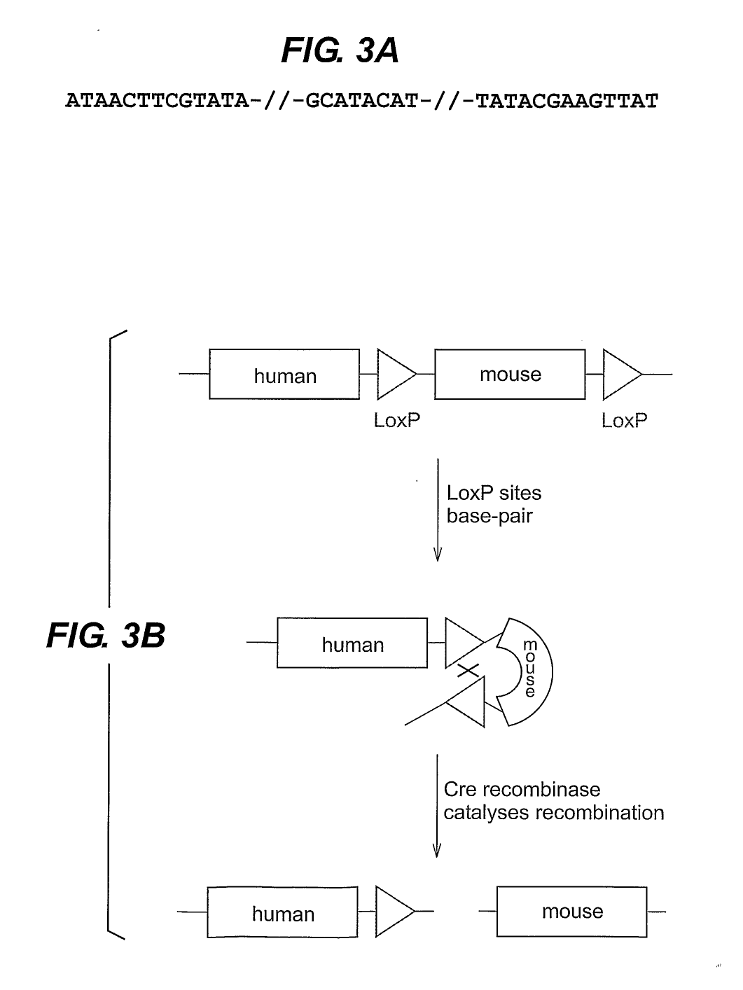

The next stage of the method of the present invention is the excision of the endogenous host gene target sequence. Excision is effected by effecting recombination between the two type I RT sites which flank the endogenous host gene target sequence. In order to effect recombination between the RT sites, the genome must be exposed to site-specific recombinase (SSR) activity, in the form of an SSR enzyme which recognises the type I RT sites. Exposure to SSR enzyme activity results in a DNA rearrangement determined by the disposition of the RT sites, which in a linear DNA molecule results in the intervening sequence being excised, or cut out. The term “SSR” refers to any protein component of any recombinant system that mediates DNA rearrangements in a specific DNA locus, including SSRs of the integrase or resolvase/invertase classes (Abremski, K. E. and Hoess, R. H. (1992) Protein Engineering 5, 87-91; Khan, et al., (1991) Nucleic acids Res. 19, 851-860; Nunes-Duby et al., (1998) Nucleic Acids Res 26 391-406; Thorpe and Smith, (1998) P.N.A.S USA 95 5505-10) and site-specific recombination mediated by intron-encoded endonucleases (Perrin et al., (1993) EMBO J. 12, 2939-2947). The mechanism through which site-specific recombination proceeds is depicted in FIG. 3b.

Following SSR mediated recombination between the two type I RT sites, a residual type I RT site remains within the host cell chromosome, at the position previously occupied by the endogenous host gene target sequence, and the endogenous host gene target sequence is excised. The endogenous host gene target sequence now exists within the cell as a free linear DNA molecule, which will be rapidly degraded by cellular exonucleases.

The next step in the method of the present invention is the provision of the heterologous replacement gene sequence, potentially as a linear nucleic acid molecule, but preferably contained within a vector of some kind such as a bacterial artificial chromosome (BAC), yeast artificial chromosome (YAC) or the like. Examples of suitable vectors are widely known in the art.

The heterologous replacement gene sequence is flanked on one side by a type I RT site, and on the other side by a type II RT site, whereby the type I RT site is the same type as the type I RT site inserted into the host cell chromosome, and the type II RT site is the same type as the type II RT site inserted into the host cell chromosome. Importantly, the type I RT site is different from the type II RT site, such that it is heterospecific, and as such cannot interact with the type I RT site. The type I RT site flanking the heterologous replacement gene sequence is positioned in the same orientation as the type I RT site on the host cell chromosome, and the type II RT site at the other flank of the heterologous sequence is positioned in the same orientation as the type II RT site on the host cell chromosome, to allow effective recombination between the pairs of corresponding RT sites in due course.

In order for SSR-mediated recombination between the nucleic acid containing the heterologous replacement gene sequence and the host cell chromosome to occur, that sequence must be brought into close proximity with the host cell chromosome. Examples of suitable targeted delivery systems will be clear to those of skill in the art and are listed above.

Under appropriate conditions, recombination between the corresponding RT sites in the nucleic acid containing the heterologous replacement gene sequence and in the host cell chromosome is effected. These recombination steps preferably occur concurrently, and facilitate the introduction of the heterologous replacement gene sequence into the host cell chromosome at the position previously occupied by the endogenous host gene target sequence. The proximal positioning of the type I and type II RT sites on the host cell chromosome leads to an increased efficiency of insertion of the heterologous replacement gene sequence compared to methods previously described in the prior art.

### Selectable Markers

Each of the type I RT sites incorporated into the host chromosome should preferably be linked to, and preferably contiguous to one or more selectable markers. These selectable markers function to allow monitoring of host cells, such as embryonic stem cells, into which the exogenous DNA has successfully integrated. According to a further aspect of the invention, each type I RT site may be contiguous with one or more selectable markers. Preferably, each type I RT site is contiguous with 2 selectable markers. Preferably, each type I RT site is contiguous with at least one positive selection cassette, wherein a positive selection cassette will allow the detection of cells which have successfully incorporated the nucleic acid sequence. More preferably, the positive selection cassette allows selection by ensuring that only cells containing the nucleic acid sequence can survive in the growth medium. Preferably, each type I RT site is contiguous with at least one negative selection cassette, wherein a negative selection cassette will allow the detection of cells which have successfully had the nucleic acid sequence excised. More preferably, the negative selection cassette allows selection by ensuring that only cells not containing the nucleic acid sequence can survive in the growth medium. Most preferably, each type I RT site is contiguous with one positive selection cassette and one negative selection cassette.

Preferably, the one or more selectable markers are positioned so that the selectable markers lie between the endogenous host gene target sequence and the type I RT site, such that they are excised with the host gene sequence in due course.

The positive selection cassette is preferably a gene encoding some kind of resistance to a chemical compound to which the growing host cells can be exposed, such as an antibiotic. Examples include use of selectable markers conferring resistance to antibiotics added to the growth medium of cells, for instance the neomycin resistance marker conferring resistance to G418, hygromycin or puromycin. Further examples involve detection using nucleic acid sequences that are of complementary sequence and which will hybridise with, the nucleic acid sequence in accordance with the previous aspects of the invention. Examples would include Southern blot analysis, northern blot analysis and PCR.

The negative selection cassette is preferably a gene conferring sensitivity to a chemical compound. For example, a thymidine kinase (TK) gene may be used, and will confer sensitivity to ganciclovir.

Within a further aspect of the invention, the selectable markers are selected from a Thymidine kinase expression cassette, a hygromycin resistance gene and a promoter-less and ATG-deficient Neomycin cassette (5′ΔNeo) (see Seibler et al., 2005, Nucl Acids Res. 33(7) e67).

Within a further aspect of the invention, one of the type I RT sites is contiguous to a Thymidine kinase expression cassette and 5′ΔNeo, and the other type I RT site is contiguous to a thymidine kinase expression cassette and a hygromycin resistance gene.

Preferably, the 5′ΔNeo sequence, that is located so as to be linked to one of the type I RT sites within the host cell chromosome, facilitates selection of cells due to the presence of a promoter and ATG within the host chromosome. The basis of this concept is to use a promoterless and ATG-deficient neomycin cassette as a marker for integration. If this integrates randomly into the genome, this cassette is inactive and does not confer G418 resistance. It can be activated only by a precise insertion into an already prepared locus which contains the promoter and the ATG. This thus provides a stringent selection process for successful integration at a correct location in the chromosome.

In one embodiment, after insertion of the deficient sequence into the chromosome, the ATG is separated from the neomycin by a loxP site. The complemented expressed neomycin sequence thus forms a fusion protein of amino acids encoded by the loxP site and the 3′ half of the neomycin cassette.

In one aspect of the invention, the heterologous replacement gene sequence is on a vector, and that vector preferably contains one or more selectable markers. Preferably, the vector contains 2 selectable markers, preferably selected from a neomycin expression cassette and a hygromycin resistance gene. The one or more selectable markers contained on the vector are preferably positioned between the type I RT site and the heterologous replacement gene sequence, and/or between the type II RT site and the heterologous replacement gene sequence. Preferably, at least one selectable marker is positioned on either side of the heterologous replacement gene sequence. More preferably one selectable marker is positioned on each side of the heterologous replacement gene sequence.

This selection system has the advantage that it is entirely directed by the selection marker genes introduced into the constructs by the experimenter. Therefore, the method of the present invention can be utilised in any embryonic stem cell, without the requirement for an initial selection pressure. This is in contrast to the method of Wallace et al, which can only be performed in an HPRT-deficient (hprt⁻) embryonic stem cell line.

### Additional RT Sites

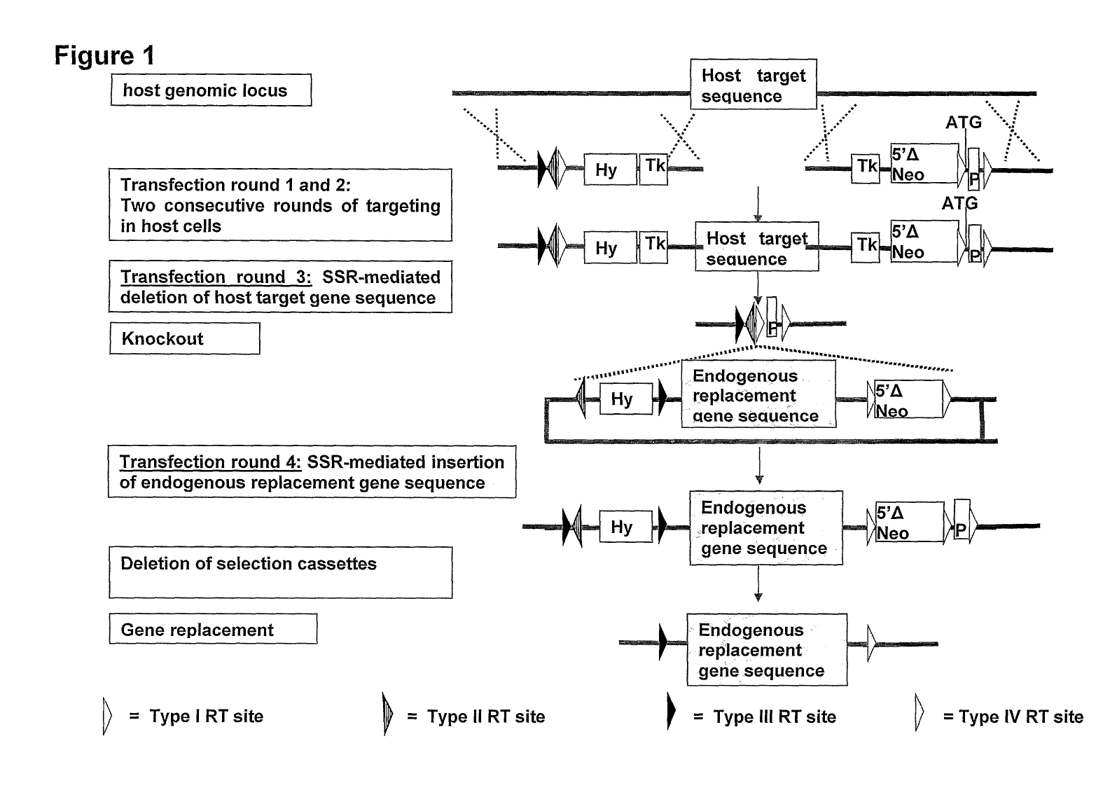

In a still further aspect of the invention, the host chromosome is modified so as to contain one or more further RT sites in addition to the pair of type I RT sites and the type II RT site. Preferably the host chromosome contains two additional RT sites as illustrated in FIG. 1. More preferably the host chromosome contains one type III RT site and one type IV RT site. These additional RT sites are incorporated into the host cell chromosome by homologous recombination in the same manner as described previously for the type I and type II RT sites. Preferably, the additional RT sites are incorporated concurrently with the type I and type II RT sites.

In this further embodiment, the type II RT site incorporated into the host chromosome is flanked by a type III RT site, such that the type II RT site is positioned between the type I RT site and the type III RT site.

In a further embodiment, the type I RT site present in the host chromosome which is not flanked proximally by a type II RT site is flanked by a type IV RT site, such that the type I RT site is positioned between the endogenous host gene target sequence and the type IV RT site.

In another aspect of the invention, the vector contains one or more further RT sites in addition to the type I RT site and the type II RT site. Preferably the vector contains two additional RT sites. More preferably the vector contains one type III RT site and one type IV RT site.

In a further embodiment, the additional RT sites are positioned within the vector so that they are flanked by the type I or type II RT site. Preferably the type III RT site within the vector is located such that the type III RT site is positioned between the type II RT site and the heterologous replacement gene sequence. More preferably, the type III RT site is positioned between the heterologous replacement gene sequence and the one or more selectable markers, such that the one or more selectable markers are positioned between the type II RT site and the type III RT site.

Preferably the type IV RT site within the vector is located such that the type IV RT site is positioned between the type I RT site and the heterologous replacement gene sequence. More preferably, the type IV RT site is positioned between the heterologous replacement gene sequence and the one or more selectable markers, such that the one or more selectable markers are positioned between the type I RT site and the type IV RT site.

Furthermore, the additional RT sites present on the vector are aligned in the same direction as the corresponding RT sites in the host cell chromosome, so as to allow their recombination together in due course. Insertion of the heterologous replacement gene sequence into the host cell chromosome through SSR mediated recombination at the corresponding type I and type II RT sites positioned on the vector and the host cell chromosome, as described above, results in concurrent insertion of the additional RT sites into the host cell chromosome

Effecting recombination between corresponding type I and type II RT sites located on the vector and in the host chromosome, to insert the heterologous replacement gene sequence into the host chromosome, results in the positioning of the one or more selection markers present on one side of the heterologous replacement gene sequence and the residual type I RT site between two type III RT sites, and in the positioning of the one or more selection markers present on the other side of the heterologous replacement gene sequence and the residual type II RT site between the two type IV RT sites.

Two separate recombination steps may then be effected between corresponding additional type III and type IV RT sites incorporated into the host chromosome. The two additional recombination steps result in the excision of portions of DNA from the host cell chromosome, which included the selection cassettes.

This is advantageous as it prevents the possibility of selectable markers having detrimental effects when they persist in the host cell chromosome. An example of such a detrimental effect is a change of the expression of genes in proximity to the selectable markers and the contribution of a selectable marker to antibiotic resistance. Further, the two additional recombination steps may result in the excision of the residual type I and type II RT sites lying between them in the chromosome.

The two additional recombination steps thus facilitate the deletion of all non-exogenous DNA, with the exception of the heterologous replacement gene sequence, and the two residual RT sites. Preferably the two residual RT sites are a residual type III RT site and a residual type VI RT site.

### RT Sites

In order to effect recombination between the RT sites, the genome must be exposed to site specific recombinase (SSR) activity, in the form of an SSR enzyme. Exposure to SSR enzyme activity results in a DNA rearrangement determined by the disposition of the RT sites, which in a linear DNA molecule results in the intervening sequence being excised, or cut out. The term “SSR” refers to any protein component of any recombinant system that mediates DNA rearrangements in a specific DNA locus, including SSRs of the integrase or resolvase/invertase classes (Abremski, K. E. and Hoess, R. H. (1992) Protein Engineering 5, 87-91; Khan, et al., (1991) Nucleic acids Res. 19, 851-860; Nunes-Duby et al., (1998) Nucleic Acids Res 26 391-406; Thorpe and Smith, (1998) P.N.A.S USA 95 5505-10) and site-specific recombination mediated by intron-encoded endonucleases (Perrin et al., (1993) EMBO J. 12, 2939-2947).

The methodology for mediating Cre/lox-mediated deletions, suitable for deleting of large fragments of DNA (200 kb to several megabases), has been described in the following papers (Li Z W, Stark G, Gotz J, Rulicke T, Gschwind M, Huber G, Muller U, Weissmann C. Generation of mice with a 200-kb amyloid precursor protein gene deletion by Cre recombinase-mediated site-specific recombination in embryonic stem cells Proc Natl Acad Sci USA. 1996 Jun. 11; 93(12):6158-62. Erratum in: Proc Natl Acad Sci USA 1996 Oct. 15; 93(21):12052; in Su H, Wang X, Bradley A. Nested chromosomal deletions induced with retroviral vectors in mice. Nat Genet. 2000 January; 24(1):92-5); Call L M, Moore C S, Stetten G, Gearhart J D. A cre-lox recombination system for the targeted integration of circular yeast artificial chromosomes into embryonic stem cells. Hum Mol Genet. 2000 Jul. 22; 9(12):1745-51).

It is to be understood that the site-specific recombination steps of the present invention can be effected in vivo or in vitro.

For in vitro recombination, the SSR corresponding to the RT site must be introduced into the altered host cell. Such introduction can occur by the introduction of the SSR protein directly into the cell, or by the introduction of an exogenous gene encoding the SSR, which is subsequently expressed. Examples of suitable targeted delivery systems for delivery of a gene encoding the SSR will be clear to those of skill in the art and include the systems described above.

In vivo recombination may be desirable if a transgenic organism has been produced, as described below. Site-specific recombination may then be effected by inducing activity of the SSR within the transgenic organism. Successful exploitation of site-specific recombination to alter genotype in living systems generally requires strategies to regulate the recombination event. This can be done by controlling expression of the recombinase mRNA, or protein (Baubonis and Sauer (1993) Nucl Acids Res. 21, 2025-2029; Sauer B, (1994) Curr Opin Biotechnol 5:521-7; Rajewsky et al., (1996) J Clin Invest 98, 600-3; Metzger and Feil, (1999) Curr. Opinions Biotechnology 10, 470-476), such that the expression pattern achieved is confined to the times and places at which these tissue specific elements are active. Expression can be controlled in a tissue-specific pattern e.g. albumin-Cre in the liver.

Researchers have used direct transfection, infection with recombinant viruses or injection of the DNA or mRNA encoding SSR protein or the protein itself (Konsolaki et al., (1992) New Biol. 4: 551-557) in order to express SSR enzymes. A more precise degree of control may be attained by regulating the activity rather than the expression of these SSR enzymes. One strategy uses fusion proteins in which a SSR enzyme is fused to the ligand binding domain (LBD) of a steroid receptor to give an SSR-LBD protein (see EP-B-0 707 599; also Logie and Stewart (1995) P.N.A.S. USA 92: 5940-5944; Brocard et al., (1997) P.N.A.S. USA 94: 14559-14563; Akagi et al., (1997) Nucleic Acids Res 25, 1766-73). This strategy relies on the application of a ligand for the steroid receptor that activates the SSR activity only when ligand is bound to the receptor moiety. The LBD of the receptor represses the activity of the SSR in the absence of a cognate ligand. Delivery of the cognate ligand relieves repression of the SSR, thus permitting recombination between RT sites.

Induction may thus be effected by inducing transcription of the SSR, inducing translation of the SSR, or removing an inhibitor from the SSR. Alternatively, an SSR may be artificially introduced into the transgenic organism. One element of the methodology is that site-specific recombination can be effected within the transgenic organism, so resulting in the excision of the endogenous host gene target sequence and the concomitant production of a transgenic organism containing the heterologous replacement gene sequence in place of the endogenous host gene target sequence.

Preferably, site-specific recombination can be effected in vivo by crossing a transgenic mouse with a deleter strain mouse. The term “deleter strain” as used herein relates to a mouse expressing the site-specific recombinase in its germline, which can be crossed with a transgenic mouse to effect excision of the mouse target gene sequence. In this manner, in vivo recombination produces offspring heterozygous for the gene of interest. Crossing the transgenic mouse with a deleter strain will thus result in the production of progeny, with cells containing the mouse chromosome altered to contain the human replacement gene sequence and the site-specific recombinase, resulting in the excision of the mouse target gene and the functional humanisation of the cells. Such a transgenic mouse will therefore be heterozygous for humanisation of the specific gene or cluster of genes.

In certain embodiments, it may be desired for the site-specific recombinase only to be expressed in a certain tissue of the recombinase strain mouse. It is known in the art that deletion of certain genes or clusters of genes may be lethal or may have sublethal phenotypic effects. Furthermore, replacing such genes with their human equivalents may not prevent lethality. In these circumstances, it may be possible to overcome any such problems of lethality by expressing the site-specific recombinase only in certain tissues, for example, the liver. This will be particularly advantageous if a specific gene is known to be essential in a certain tissue, as expression of the site-specific recombinase in this manner allows the mouse gene to persist in those tissues.

Within this aspect of the invention, the SSR may be albumin-Cre. Albumin-Cre is a specific variant of the SSR Cre which acts on the RT site LoxP. Albumin-Cre is expressed only in the liver, and will therefore allow the mouse target sequence to persist in all tissues except the liver, overcoming possible problems of lethality, whilst providing a functionally humanised liver.

Ultimately, two heterozygous mice produced according to the methodology above may be crossed to produce a transgenic mouse that is homozygous for the human allele of the gene or genes of interest. Crossing two heterozygous transgenic mice will produce a proportion of progeny that are homozygous for the humanised allele.

In a further embodiment of the invention the transgenic non-human animal is produced de novo so as to include all of the aforementioned features, by the methods as hereinafter disclosed.

It is also possible that the site-specific recombination event be effected in a somatic cell which could then be used as a nuclear transfer donor cell in order to make a colony of cloned mice according to the methodology of WO00/51424 or a variation thereof.

In another embodiment of the invention a transgenic animal according to the present invention is produced by crossing. For example, a mouse which still includes unwanted sequences between RT sites could be crossed with mouse expressing an SSR enzyme.

In a further embodiment of the invention the transgenic mouse is produced de novo so as to include all of the aforementioned features, by the methods as hereinafter disclosed.

Within a preferred embodiment of the invention, none of the type I RT site, the type II RT site, the type III RT site, and the type IV RT site are the same, such that each type of RT site is heterospecific with respect to each of the other types of RT sites, and as such that none of the RT sites can interact with another RT site of a different type.

Preferred recombinase proteins are selected from the group consisting of: FLP recombinase, Cre recombinase, Dre recombinase, R recombinase from Zygosaccharomyces rouxii plasmid pSR1, a recombinase from the Kluyveromyces drosophilarium plasmid pKD1, a recombinase from the Kluyveromyces waltii plasmid pKW1, TrpI from the Bacillus transposon Tn4430, any component of the λ Int recombination system, phiC31, any component of the Gin recombination system, or variants thereof. The list is provided by way of example only, and is not intended to be limiting.

Preferably, the site-specific recombination sites are chosen from loxP, lox5171, lox511, F3 and FRT.

In one aspect of the invention, the type I RT sites is loxP. In another aspect of the invention, the type II RT site is lox5171. In another aspect of the invention, the type III RT site is FRT. In a further aspect of the invention, the type IV RT site is F3. The skilled reader will understand that these RT sites can be interchanged such that lox1517 is type I, loxP is type II and so on. Furthermore, any other heterospecific mutant of any of the RT sites could be used. For examples, any heterospecific mutant of loxP, e.g. lox511, could be used.

### Vectors

It is preferred that the heterogenous replacement gene sequence is introduced into the host cell on a vector. As indicated above, the vector may preferably be a normal cloning vector, a Bacterial Artificial Chromosome or a Yeast Artificial Chromosome. Preferably, the vector is a BAC.

As described previously, the optional vector contains the heterologous replacement gene sequence, which may comprise one or more gene(s) or segments of genes. The heterologous replacement gene sequence may also comprise the regulatory regions associated with the one or more gene(s) of segments of genes. The vector also comprises a type I RT site and a type II RT site, which flank the heterologous replacement gene sequence, and one or more selectable markers.

### The Endogenous Host Gene Target Sequence

The host cell of the present invention may be any prokaryotic or eukaryotic cell in which it is possible for homologous recombination to take place, including bacteria, yeast, animal and plant cells. However, the host cell is preferably a eukaryotic cell, more preferably a stem cell, such as an ES cell or an iPS cell (see Takahashi et al., Nat Protoc. 2007; 2(12):3081-9; Yamanaka, Cell Prolif. 2008 February; 41 Suppl 1:51-6). Within one aspect of the invention, the host embryonic stem cell is a mammalian stem cell, such as a mammalian ES cell. Within a further aspect of the invention, the mammalian embryonic stem cell is a mouse embryonic stem cell.

The invention may be implemented using any one of a number of genes, as will be clear to those of skill in the art. There is no technical limitation to the type of genes that may be exchanged between host cell target and heterologous replacement. The invention is illustrated herein using a P450 gene cluster, in which the mouse Cyp3A, Cyp2C, or Cyp2D cluster is replaced by the human equivalent cluster. P450 genes are interesting candidates for humanisation, particularly on a null background, since such systems allow the human metabolic response to drug molecules to be assessed in the absence of interference from competing murine systems. The genes are often very large, although they are generally clustered together in families of similar function. Accordingly, the methods of the invention lend themselves particularly well to the study of these humanised systems.

Preferably, therefore, the expression product of the host cell target gene retains the same, similar, equivalent or identical function as the heterologous replacement gene. The genes may be functionally equivalent, and/or structurally homologous. For example, the host cell target gene and heterologous replacement gene may share a degree of homology. Preferably, such homology will be greater than 30%, greater than 40%, greater than 50%, greater than 60%, greater than 70%, greater than 80%, greater than 90%, or even greater than 95%.

As will be apparent to one skilled in the art, the endogenous host gene target sequence excised from the host cell chromosome will be defined by the position of the type I RT sites, which recombine to excise the DNA segment contained between them. The position of the type I RT sites is dependant upon the location of the regions of homology between the host cell chromosome and the exogenous DNA segments containing the type I RT sites. Therefore, it will be apparent to one skilled in the art that any number of genes or gene segments can be excised from the host cell chromosome using the method of the present invention.

Furthermore, the regulatory regions associated with the endogenous host gene target sequence can be excised, or can remain in the host cell chromosome, depending upon the position of the type I RT sites. If the regulatory regions associated with the endogenous host gene target sequence remain within the host cell chromosome, they may become operatively linked to the heterologous replacement gene sequence. The advantage of this approach is that the endogenous gene expression pattern will be seen, and gene expression will be controlled in the same manner as it is in the unmodified host cell. This may have important implications for genes which are not normally expressed in the host cell.

### The Heterologous Replacement Gene Sequence

The heterologous replacement gene sequence may preferably comprise cDNA, genomic DNA, or a mixture of the two. Genomic DNA is advantageous in many circumstances because the fidelity of splicing will be retained. However, it may only be necessary to retain those introns where the majority of splice events take place, such that the remainder of the sequence can be cDNA. This can simplify the cloning process, particularly where the genomic DNA comprises large introns; in such cases the larger introns may not be included provided that splice isoforms are not coded for in this area of the genomic DNA.

It will be understood that the heterologous replacement gene sequence is defined by the position of the type I and type II RT sites present in the vector, with the entire nucleic acid sequence between these two RT sites being inserted into the host cell chromosome, upon recombination with the corresponding RT sites present in the host cell chromosome. The heterologous replacement gene sequence can therefore correspond to a gene segment, a whole gene, or a number of genes.

The heterologous replacement gene sequence may include regulatory sequences associated with the gene(s), or gene segment. These regulatory sequences would therefore be inserted into the host cell chromosome as part of the heterologous replacement gene sequence. The regulatory sequences may be the regulatory sequences normally associated with the heterologous gene(s) or gene segment, and the gene(s) or gene segment would remain under the control of the regulatory sequences which normally control the gene(s) or gene segment. This may be advantageous as it would allow the heterologous replacement gene sequence to be expressed in the host cell in the same manner as it would normally be expressed. However, as described above, it may also cause expression problems.

In another embodiment the regulatory sequences associated with the gene(s) or gene segment may be heterologous sequences normally not associated with the gene(s) or gene segment included within the heterologous replacement gene sequence. Within this embodiment, the regulatory sequences may be tissue specific regulatory sequences, including but not limited to regulatory sequences including the albumin promoter, the apoE promoter or the villin promoter.

In one aspect of the invention, the heterologous replacement gene sequence is a mammalian gene sequence.

In a further aspect of the invention, the mammalian replacement gene sequence is a human replacement gene sequence.

### Provision of Knockout Lines

In the course of the method of the present application, cell lines may be generated that contain a knockout of a particular endogenous gene or gene cluster. In one embodiment, the endogenous gene or cluster of genes is a member of the Cytochrome P450 family. Examples include the Cyp3a, Cyp2c and Cyp2d clusters.

Preferably the knockout cell line is stable. By “stable” is meant that the knockout cell line is able to be maintained in a viable form in cell culture for a minimum of 1 week. In other embodiments the knockout cell line is able to be maintained in a viable form for a minimum of 2 weeks, 3 weeks, 4 weeks, 1 month, 6 months, 1 year, 2 years or more. Taken differently, a stable cell line is one which can be passaged at least 5 times, at least 10 times, at least 20 times, at least 30 times, at least 50 times, at least 100 times, at least 200 times or more whilst remaining viable.

In one embodiment, the cell line used to produce the knockout cell line is a mammalian cell line. In another embodiment, the mammalian cell line is a mouse cell line. In a further embodiment the mouse cell line is a mouse stem cell line. In yet a further embodiment the mouse stem cell line is a mouse ES cell line.

The production of a stable knockout cell line is advantageous because such a pre-prepared knockout cell line can be used for the insertion of a heterologous replacement gene sequence according to the method described above. The pre-prepared knockout cell line allows fewer steps to be performed in ES cells at the time of insertion of the heterologous replacement gene sequence, and will therefore increase the efficiency of transformation, and the frequency of correctly targeted clones.

Generation of Humanised Cell Lines from Knockout Cell Lines

The knockout cell lines described above may be used as the host cell line for the insertion of a heterologous replacement gene sequence or gene cluster according to the method described above. In one embodiment the heterologous replacement gene sequence is a mammalian heterologous replacement gene sequence or gene cluster. In another embodiment, the mammalian heterologous replacement gene sequence is a human heterologous replacement gene sequence or gene cluster. In a further embodiment, the human heterologous replacement gene sequence encodes a member of the Cytochrome P450 family. The member of the Cytochrome P450 family may be a CYP3A, a CYP2C or a CYP2D gene or gene cluster. Examples of CYP3A, CYP2C and CYP2D genes are CYP3A4, CYP3A5, CYP2C9, CYP2C19 or CYP2D6.

In a preferred embodiment, the knockout cell line used as the host cell line for the insertion of a heterologous replacement gene sequence contains a knockout of the gene or gene cluster which corresponds to the gene or gene cluster contained within the heterologous replacement gene sequence.

The heterologous replacement gene sequence used for insertion into a knockout cell line may be the same heterologous replacement gene sequence described above. In one embodiment, the heterologous replacement gene sequence may contain regulatory elements associated with the gene or gene cluster. In one embodiment, such regulatory elements are endogenous to the gene or gene cluster contained within the heterologous replacement gene sequence. In another embodiment, the regulatory elements may be tissue-specific regulatory elements. Examples of tissue-specific regulatory elements are the albumin, apoE and villin promoters.

### Transgenic Organisms

Within another aspect of the invention, there is provided a transgenic organism produced by a method of any one of the embodiments of the invention described above. Such an organism contains a heterologous replacement gene sequence at the position previously occupied by the endogenous host gene target sequence, and the corresponding endogenous host gene target sequence has been deleted.

Within a further aspect of the invention, the transgenic organism is a transgenic mammal, and the deleted endogenous host gene target sequence is a mammalian gene target sequence.

Within a further aspect of the invention, the transgenic mammal is a transgenic mouse, and the deleted endogenous host gene target sequence is a mouse gene target sequence.

Within a further aspect of the invention, the heterologous replacement gene sequence is a mammalian heterologous replacement gene sequence, and within a further aspect of the invention, the heterologous replacement gene sequence is a human replacement gene sequence.

Within a further aspect of the present invention, altered stem cells such as ES cells of the invention containing the heterologous replacement gene sequence, may be inserted into a blastocyst. Conventionally, blastocysts are isolated from a female mammal, of corresponding species to the embryonic stem cell, about 3 days after it has mated. It is to be understood that up to 20 altered embryonic stem cells may be simultaneously inserted into such a blastocyst, preferably about 16. Through insertion of altered embryonic stem cells into the blastocyst, the embryonic stem cell will become incorporated into the developing early embryo, preferably by its transplantation into a pseudo-pregnant mammal which has been induced so as to mirror the characteristics of a pregnant mammal. According to this methodology, the blastocyst, containing the altered embryonic stem cell, will implant into the uterine wall of the pseudo-pregnant mammal and will continue to develop within the mammal until gestation is complete. The altered embryonic stem cell will proliferate and divide so as to populate all tissues of the developing transgenic mammal, including its germ-line.

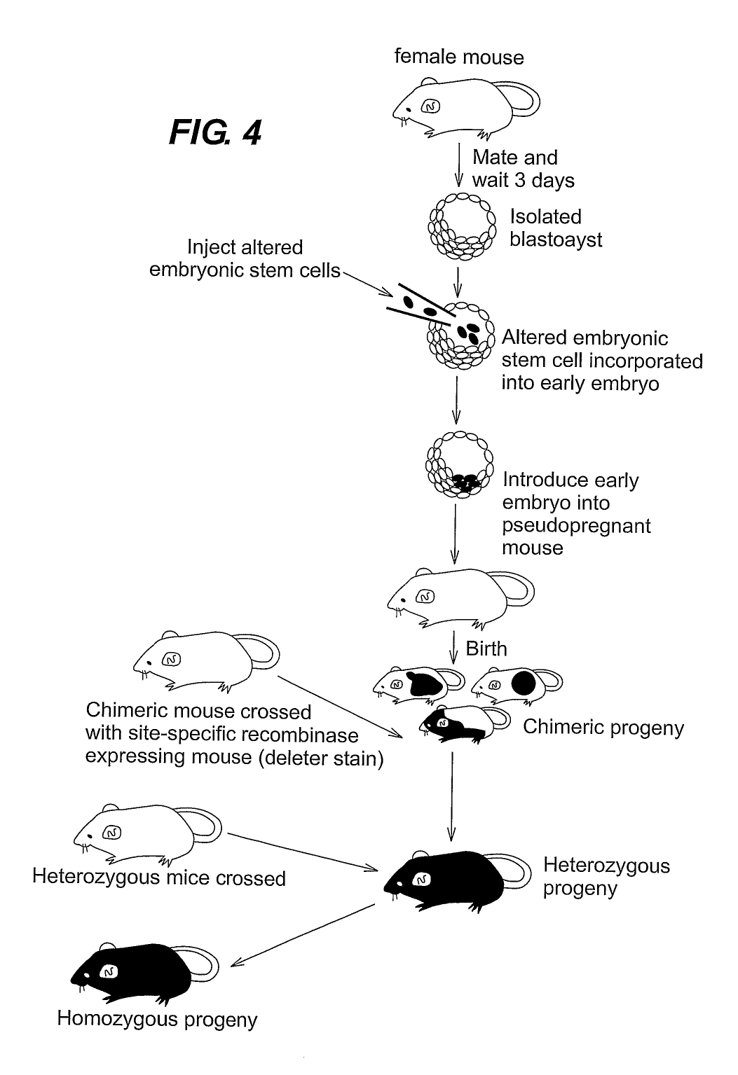

In one aspect of the methodology, the created transgenic mammal may be a chimera, containing altered and non-altered cells within each somatic tissue and within the germ-line. Preferably, the pseudo-pregnant mammal is a pseudo-pregnant mouse, and the altered cell is a mouse embryonic stem cell, as depicted in FIG. 4.

In a further aspect of the methodology, the chimeric transgenic mammal generated by the method described above may be crossed with another chimeric transgenic mammal generated by the method described above, and the resulting progeny tested to identify a mammal homozygous for the inserted heterologous gene replacement sequence. Methods which may be used to identify a mammal homozygous for the inserted heterozygous replacement gene sequence will be apparent to a person skilled in the art. By way if illustration and not limitation, homozygotes may be identified by taking the tail tip of the mammal, PCR amplifying the section of the genome of interest, and sequencing the gene cluster of interest. Alternatively, a probe specific for the heterologous gene replacement sequence may be used to identify homozygotes.

Generation of Single or Multiple Humanised Mammal Lines Using Cell Lines Produced from a Mammalian Knockout Cell Line

Within another embodiment of the invention, there is provided a single or multiple humanised mammal line produced according to any of the methods described above, wherein the host cell is a mammalian knockout cell line, as described above. Such an organism contains a heterologous replacement gene sequence at the position previously occupied by the endogenous host gene target sequence, before the knockout cell line was produced.

In one embodiment, the humanised mammal is a mouse.

Within a further aspect of the present invention, humanised stem cells such as ES cells generated from knockout cell lines produced according to the invention and containing the heterologous replacement gene sequence, may be inserted into a blastocyst. Conventionally, blastocysts are isolated from a female mammal, about 3 days after it has mated. It is to be understood that up to 20 altered embryonic stem cells may be simultaneously inserted into such a blastocyst, preferably about 16. Through insertion of altered embryonic stem cells into the blastocyst, the embryonic stem cell will become incorporated into the developing early embryo, preferably by its transplantation into a pseudo-pregnant mammal which has been induced so as to mirror the characteristics of a pregnant mammal. According to this methodology, the blastocyst, containing the altered embryonic stem cell, will implant into the uterine wall of the pseudo-pregnant mammal and will continue to develop within the mammal until gestation is complete. The altered embryonic stem cell will proliferate and divide so as to populate all tissues of the developing transgenic mammal, including its germ-line.

In one aspect of the methodology, the created transgenic mammal may be a chimera, containing altered and non-altered cells within each somatic tissue and within the germ-line.

In one aspect of the invention, the chimeric transgenic mammal may be humanised for a gene or gene cluster belonging to the Cytochrome P450 family. In another aspect, the Cytochrome P450 family may be a CYP3A, CYP2C or CYP2D gene or gene cluster. In a further aspect, the CYP3A, CYP2C or CYP2D gene may be CYP3A4, CYP3A5, CYP2C9, CYP2C19 or CYP2D6. In another aspect, the chimeric transgenic mammal may contain the human CYP3A4, CYP3A5, CYP2C9, CYP2C19 or CYP2D6 gene cluster under the control of a tissue specific promoter. In a further aspect, the tissue specific promoter may be the albumin, apoE or villin promoter. As described in more detail above, this may be advantageous for genes or gene clusters, deletion of which may be lethal, or have sub-lethal phenotypic effects in certain tissues.

In a further aspect of the methodology, the chimeric transgenic mammal generated by the method described above may be crossed with another chimeric transgenic mammal generated by the method described above, and the resulting progeny tested to identify a mammal homozygous for the inserted heterologous gene replacement sequence. Methods which may be used to identify a mammal homozygous for the inserted heterozygous replacement gene sequence will be apparent to a person skilled in the art. By way of illustration and not limitation, homozygotes may be identified by taking a tissue sample, such as a tail tip from the mammal, PCR amplifying the section of the genome of interest, and sequencing the gene cluster of interest. Alternatively, a probe specific for the heterologous gene replacement sequence may be used to identify homozygotes.

In a further embodiment, chimeric or homozygous humanised mammals which are humanised for different genes or gene clusters may be crossed in order to generate multiple humanised mammal lines. In one embodiment, one or more of a Cyp3a knockout humanised for CYP3A4, a Cyp3a knockout humanised for CYP3A5, a Cyp2c knockout humanised for CYP2C9 a Cyp2c knockout humanised for CYP2C19, or a Cyp2d knockout humanised for CYP2D6 may be crossed. In another embodiment, two, three, four or five of the humanised mammals may be crossed. In a further embodiment, one or more of the human gene clusters may be under the control of a tissue specific promoter. In yet a further embodiment, two, three, four or five of the human gene clusters may by under the control of a tissue specific promoter. In yet another embodiment, one or more of the human gene clusters may be under the control of the albumin, apoE or villin promoters. In a still further embodiment, two, three, four or five of the human gene clusters may be under the control of the albumin, apoE or villin promoters.

In a further aspect of the invention, crossing one or more of the chimeric or humanised mammals which are humanised for different genes or gene clusters may result in the production of a double, triple, quadruple, or quintuple humanised mammal line. As described above for production of single humanised mammal lines, further crossing and testing may be required to produce a mammal line homozygous for the double, triple, quadruple or quintuple humanisation.

In a further embodiment, a quadruple humanised mammal line is produced, wherein the mammal line has the endogenous Cyp3a and Cyp2c gene clusters knocked out, and the human CYP3A4, CYP3A5, CYP2C9 and CYP2C19 genes inserted. In another embodiment, one or more of the recited human genes are under the control of a tissue specific promoter. In yet a further embodiment, two, three or four of the human genes may by under the control of a tissue specific promoter. In yet another embodiment, one or more of the human genes may be under the control of the albumin, apoE or villin promoters. In a still further embodiment, two, three or four of the human genes may be under the control of the albumin, apoE or villin promoters.

This approach to mammal humanisation is advantageous because it allows the production of a quadruple humanised mammal line using pre-prepared knockout mammal ES cells, and therefore requires substantially less effort than previous methods used for the production of a quadruple humanised mammal line. In fact, the number of steps required to produce a quadruple humanised mammal line using this method is equivalent to the number of steps required to generate a double humanised mammal line using conventional methods. This reduction in the number of steps will increase the efficiency of humanised mammal line production.

Furthermore, this approach can be used with different polymeric variants of human Cyp gene clusters in order to cover all alleles of the Cyp gene cluster present in the human population.

In addition, this approach can be used to generate a multiple humanised mammal line which is humanised for a gene(s) or gene cluster different from genes of the Cytochrome P450 gene family. Examples of such genes include PXR and CXR.

### EXAMPLES

### Example 1

### Cyp3a Cluster Knockout

Construction of Cyp3a cluster targeting vectors

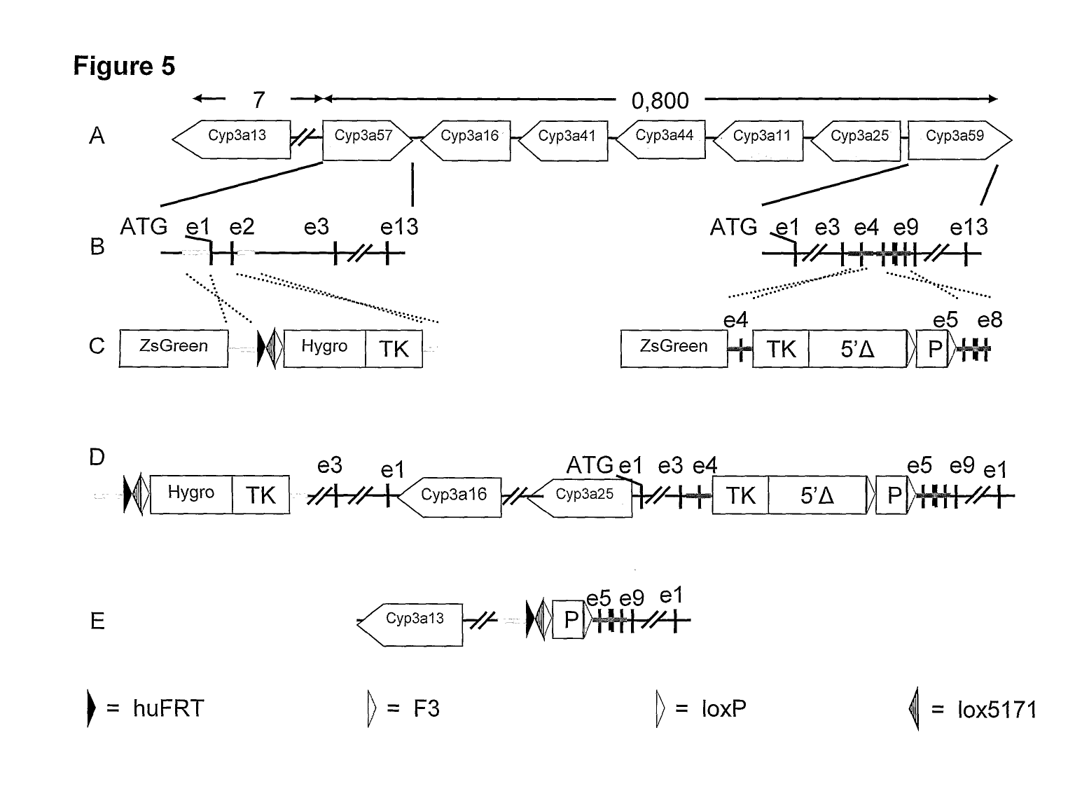

A first basic targeting vector (Cyp3a57) containing a Hygromycin, Thymidine Kinase (TK) and ZsGreen expression cassette, and a loxP, lox5171 and fit site was constructed in pBluescript (pBS). A 5.5 kb genomic sequence immediately upstream of the translational start site of the mouse Cyp3a57 gene and a 3.3 kb fragment located within intron 2 of Cyp3a57, both used as targeting arms for homologous recombination, were obtained by ET-cloning, as illustrated in Zhang et al., 1998 (Zhang, Y., Buchholz, F., Muyrers, J. P., and Stewart, A. F. 1998. A new logic for DNA engineering using recombination in Escherichia coli. Nat Genet 20:123-128.), and subcloned into the basic targeting vector as depicted in FIG. 5C.

A second basic targeting vector (Cyp3a59) containing an ATG-deficient Neomycin (5′Δ Neo), a TK and a ZsGreen expression cassette, and a loxP and f3 site was constructed in pBluescript (pBS). The translational start ATG and the corresponding promoter is separated from the 5′Δ Neo cassette in frame by the loxP site, such that additional amino acids encoded by the loxP site are fused to the N-terminus of Neomycin giving rise to a functional protein resulting in G418 resistance upon expression. A 4.3 kb genomic sequence comprising exon 4 of the mouse Cyp3a59 gene and a 5.8 kb fragment comprising exons 5-8 of Cyp3a59, both used as targeting arms for homologous recombination, were obtained by ET-cloning as illustrated in Zhang et al., 1998, and subcloned into the basic targeting vector as depicted in FIG. 5C.

### Generation and Molecular Characterisation of Targeted ES Cells

Culture and targeted mutagenesis of ES cells were carried out as previously described in Hogan et al., 1994 (Hogan, B. L. M., Beddington, R. S. P., Costantini, F., and Lacy, E. 1994. Manipulating the mouse embryo: a laboratory manual. New York: Cold Spring Harbour Press.).

The targeting vector (Cyp3a57) was linearised with Not I and electroporated into a C57BL/6 mouse ES cell line. Of 360 hygromycin resistant and fluorescence negative ES cell colonies screened by standard Southern blot analyses, 1 correctly targeted clone (B-G12) was identified, expanded and further analysed by Southern blot analyses with different suitable restriction enzymes and 5′ and 3′ external probes and an internal hygromycin probe. This clone was confirmed as correctly targeted at both homology arms and without additional random integrations (data not shown).

The second targeting vector (Cyp3a59) was linearised with Not I and electroporated into the correctly targeted Cyp3a57 ES clones B-G12 described above. Of 271 G418 resistant and fluorescence negative ES cell colonies screened by standard Southern blot analyses, 1 correctly targeted clone (A-B5) was identified, expanded and further analysed by Southern blot analyses as described above. This clone was confirmed as correctly targeted at both homology arms and without additional random integrations (data not shown).

These targeting reactions resulted in the Cyp3a gene cluster being flanked on one site by the Cyp3a57 targeting vector sequence, and on the other side by the yp3a59 targeting vector sequence, as illustrated in FIG. 5D.

Cre-Mediated In Vitro Deletion of the Cyp3a Cluster in Double Targeted ES Cells

For Cre-mediated deletion of the Cyp3a Cluster in the double targeted ES cell, 1×10⁷ ES cells derived from clone A-B5 (see above) were electroporated with the Cre-expression plasmid pCAGGScrepA as previously described in Seibler et al., 2005 (Seibler J, Kuter-Luks B, Kern H, Streu S, Plum L, Mauer J, Kuhn R, Bruning J C and Schwenk F (2005) Single copy shRNA configuration for ubiquitous gene knockdown in mice. Nucleic Acids Res 33(7):e67.) and were plated at 1 and 5×10⁵ cells, respectively, on 10 cm dishes and selected with 2 μM Ganciclovir (Calbiochem, Germany). Approximately 100 clones survived this selection, pointing to targeting of Cyp3a57 and Cyp3a59 on the same allele in clone A-B5 and a successful deletion of the mouse cluster as indicated by the loss of the TK-expression cassette conferring resistance to Ganciclovir. Resistant clones were transferred to individual wells of a 96-well plate, expanded and further analysed for deletion of the Cyp3a gene cluster by PCR with the primers 5′-GACATTGACATCCACTTTGCC-3′ and 5′-GGGAGGGAAACTTGGAGG-3′. Both primers are depicted in FIG. 5E as black arrows and only the Cre-mediated deletion of the Cyp3a Cluster brings them into close enough proximity on the chromosome to give rise to a 319 bp fragment detected by PCR. 7 of 8 Ganciclovir resistant ES cell clones analysed by PCR showed the expected band of 319 bps, confirming the successful deletion of the Cyp3a Cluster in those clones. The schematic structure of the Cyp3a cluster deleted mouse chromosome is shown in FIG. 5E.

### Example 2

### Cyp3a Cluster Humanisation

### Construction of the Modified Human BAC

The modified human Bacterial Artificial Chromosome (BAC) was generated by two seperate ET cloning steps, which introduced the required selection cassettes and site specific recombination sites in the BAC.

### Generation and Molecular Characterisation of Humanised ES Cells

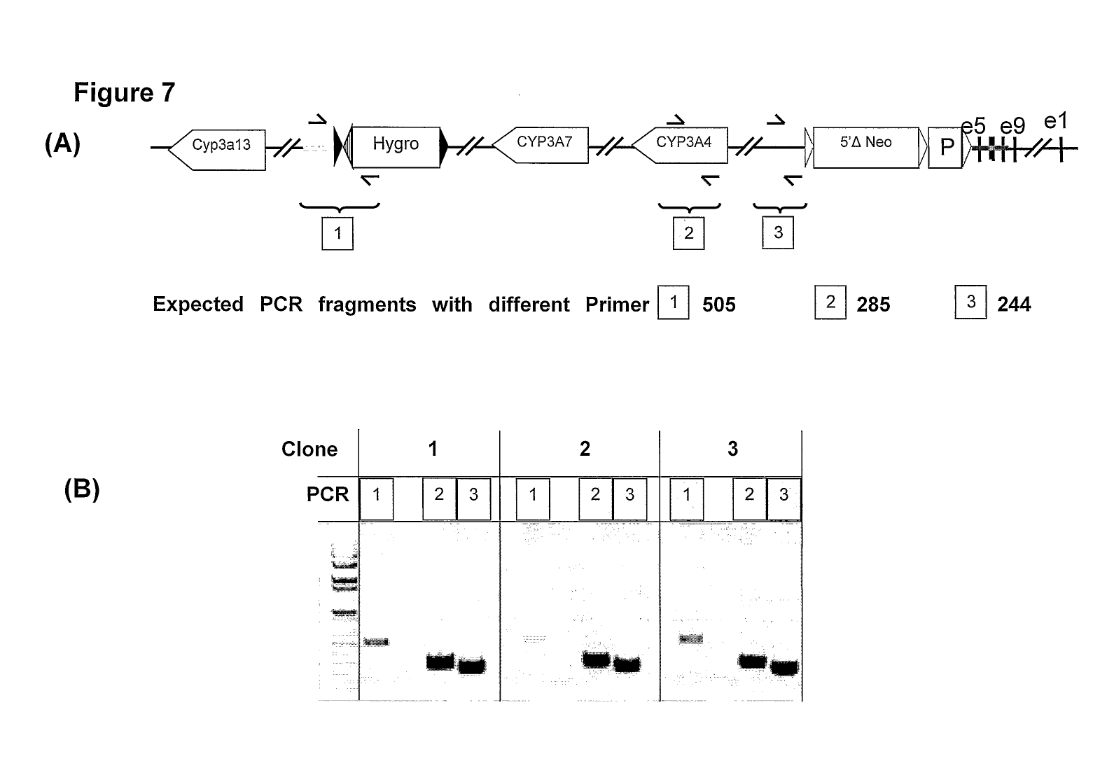

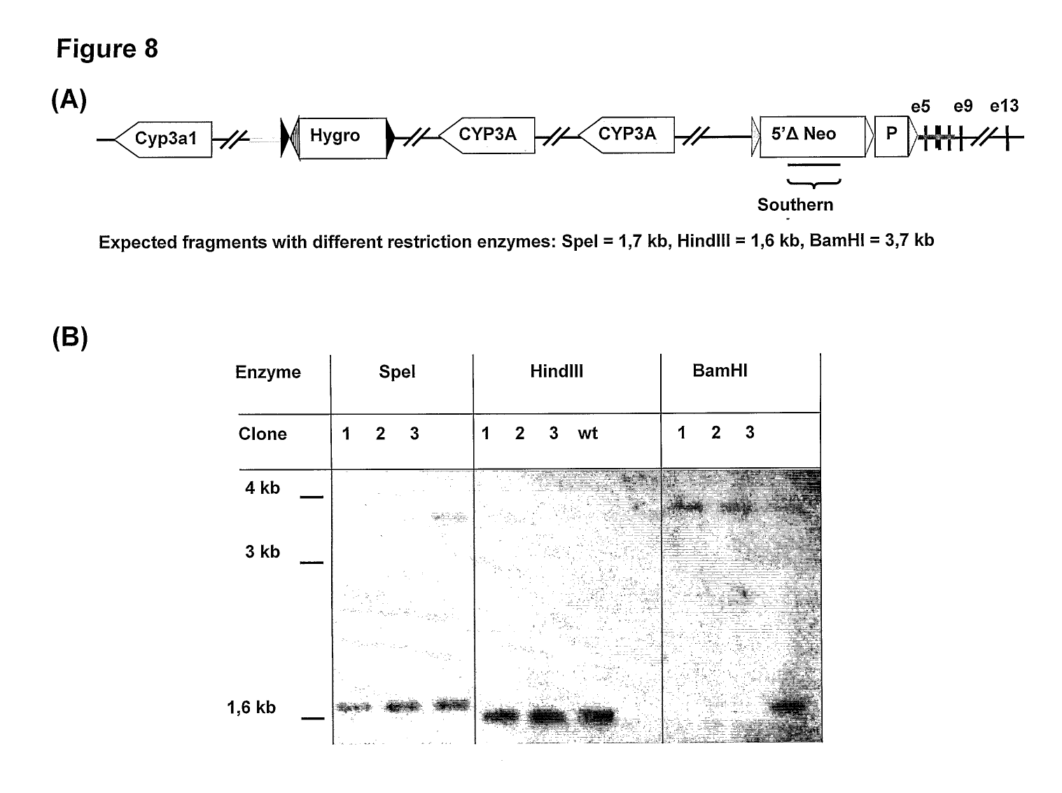

Culture and targeted mutagenesis of ES cells were carried out as described in example 1. The modified human BAC and the Cre-expression plasmid pCAGGScrepA as described in Example 1, were electroporated into 1×10⁷ Cre-deleted ES cells from the parental clone A-B5 described in Example 1. Subsequently, the electroporated ES cells were plated at 1 and 5×10⁵ cells, respectively, on 10 cm dishes and selected with G418. 7 clones survived this selection, pointing to a successful recombination at the loxP sites. As the Neomycin cassette in the human BAC is promoterless and truncated at the 5′ end, G418 resistance can only be obtained by a base pair precise integration via the loxP site. Of the 7 G418 resistant clones, 3 were expanded and further analysed by PCR and Southern blot analyses. All three clones were confirmed as correctly targeted at both ends of the human BAC, as shown in FIG. 7B, one of the 3 clones had an additional integration, as shown in FIG. 8B.

### Example 3

### Analysis of hCYP3A4/3A7_Cyp3a KO Mice

The following example is included to allow comparison between a Cypa3a knockout mouse line and a hCYP3A4/3A7 Cyp3a knockout mouse line produced according to the method of the invention. Data relating to a hCYP3A4 Cyp3a knock out mouse line are produced according to the “two-step cluster deletion and humanisation” strategy, and are included for comparison only. This method does not form part of the present invention.

Generation of hCYP3A4/3A7 Cyp3a Knockout Mice

In order to generate ES cell clones with a genomic swap of mouse Cyp3a with human CYP3A genes, the BAC clone RP11-757A13 (ImaGenes GmbH, Robert-Rossle-Str. 10, 13125 Berlin, Germany, ImaGenes Clone ID: RPCIB753A13757Q) was modified by red/ET recombineering, such that the existing lox sites in the BAC are replaced with appropriately located loxP and lox5171 sites and a hygromycin and 5′ deficient neomycin selection cassette were introduced. This allowed the insertion of the modified BAC via Cre-mediated recombination at the corresponding lox sites in the prepared Cyp3a deleted ES cell clones, as described above. and the selection of correctly targeted clones with high stringency by the complementation of the deficient neomycin cassette with the promoter and ATG remaining at the deleted Cyp3a locus. In addition, heterospecific flipase recombinase (Flp) recognition sites frt and f3 were introduced into the BAC enabling the subsequent removal of the hygromycin and neomycin selection cassettes in vivo by Flp-mediated recombination and a polyA motif was used to terminate any potential transcription initiated from the endogenous mouse Cyp3a57 promoter, which has not been deleted.

Cyp3a-deleted subclones derived from the parental clone A-B5 were used to insert the modified BAC carrying human CYP3A4 and CYP3A7 by Cre-mediated recombination. For this purpose, 1×10⁷ cells were electroporated under standard conditions with approximately 30 μg of supercoiled BAC DNA and 12 μg of the Cre-expression plasmid pCAGGScrepA as previously described (40) and selected with G418. Seven G418 resistant ES cell clones were obtained after the electroporation procedure. Three of the clones were expanded and further analysed by PCR and Southern blot with different suitable restriction enzymes, 5′ and 3′ external probes, and an internal neomycin probe. All three clones were confirmed as correctly recombined at both lox sites and didn't carry additional random integrations (data not shown). In addition, the CYP3A4 exons in the ES cell clone used to generate hCYP3A4/3A7 Cyp3a knockout mice were sequenced and it was verified that the coding region is in agreement with the accepted reference sequence (http://www.cypalleles.ki.se/cyp3a4.htm).

### Catalytic Activity Assays

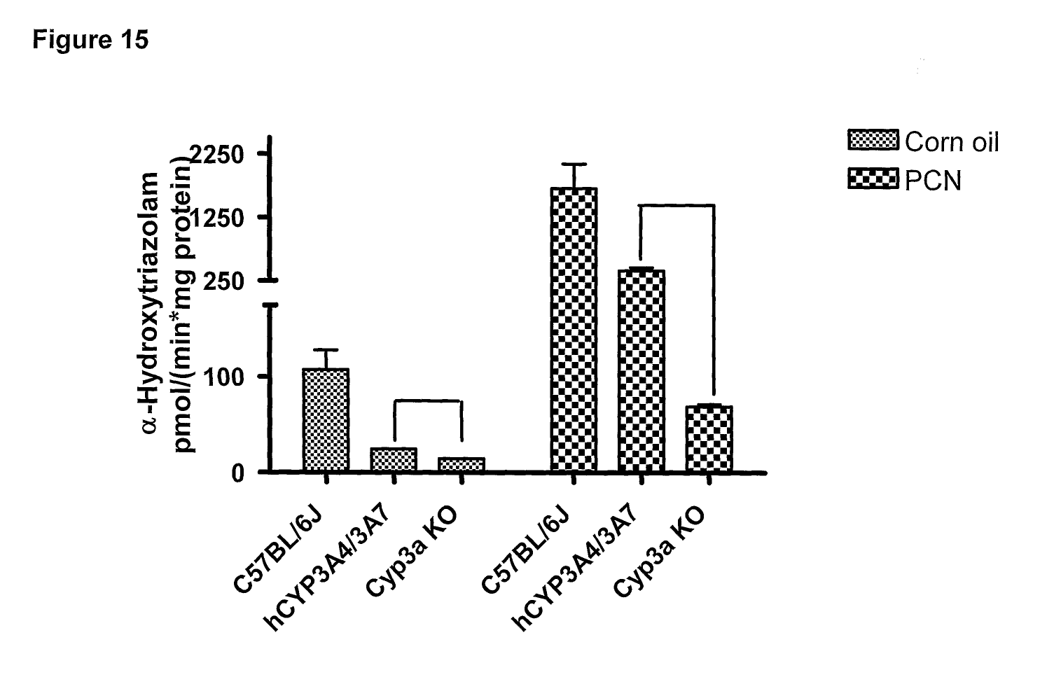

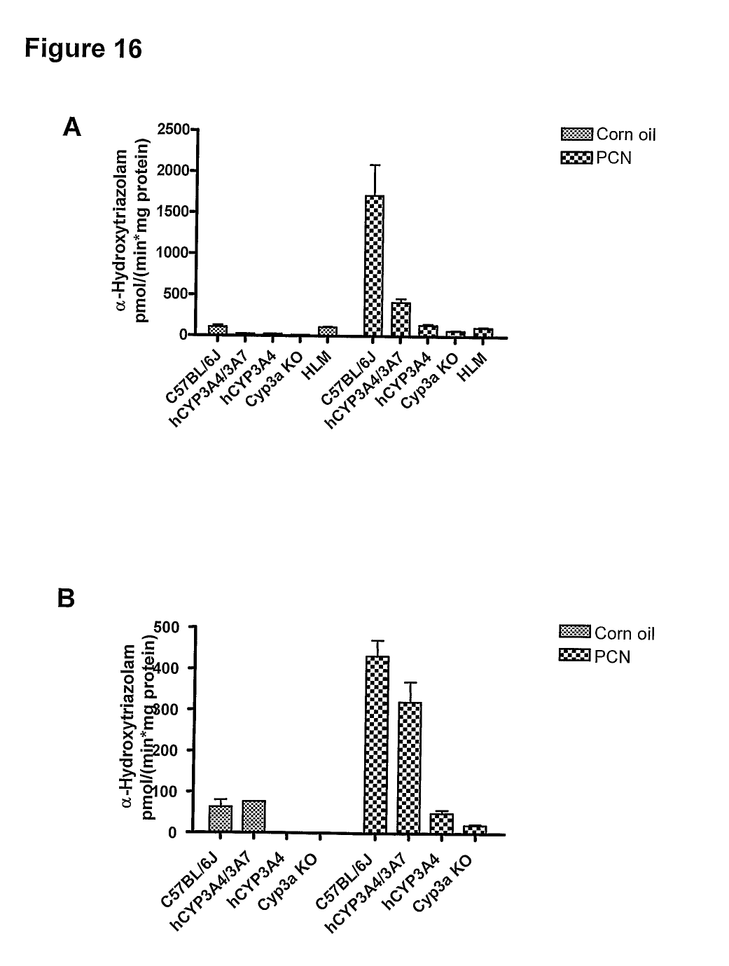

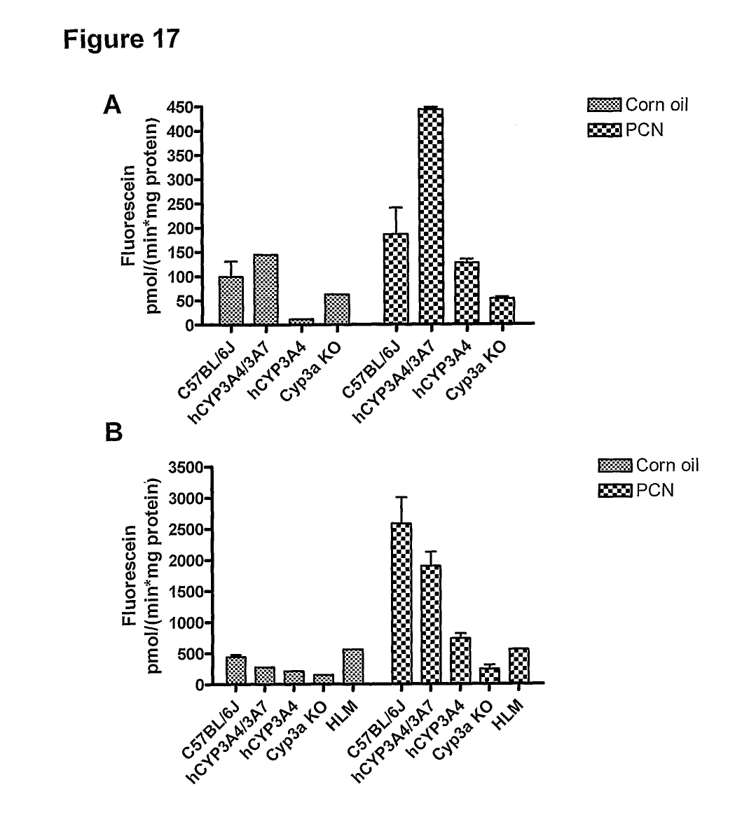

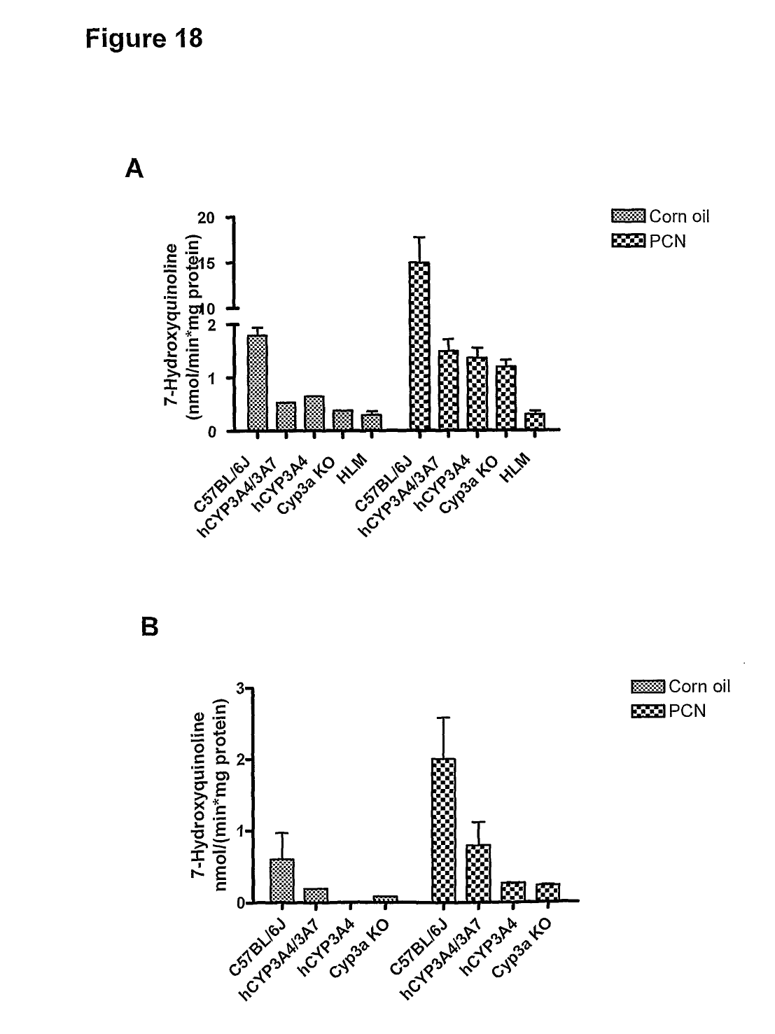

3 homozygous male mice per strain were used throughout. Two mice were administered with 5-Pregnen-3β-ol-20-one-16α-carbonitrile (PCN) (100 mg/kg/2 daily doses/IP) and one mouse was given the vehicle (corn oil). Catalytic activity was assessed using triazolam oxidation, DBF oxidation and BQ oxidation. Wild type (WT) and Cyp3a KO animals were included as controls (n=3 for WT, pooled; n=1-2 for Cyp3a KO). Animals were sacrificed 24 hrs post final dose. Liver and duodenal microsomes were analysed for CYP3A4 expression and catalytic activity. The results of this study are shown in Table 1, and in FIGS. 15-18.

In vitro oxidation of 7-benzyloxyquinoline (7-BQ) by liver and intestinal microsomes of theCYP3A4 humanised animals did not demonstrate a significant difference compared to the microsomes from Cyp3a knockout mice. This was not consistent with the Western blotting data, which suggested expression of CYP3A4 protein in both liver and small intestine of the humanised strains, particularly in the samples from PCN treated animals. In addition, the rate of 7-BQ oxidation by pooled human liver microsomes was notably lower than the reaction rate catalysed by liver microsomes from the control C57BL16J mice. Therefore, an alternative CYP3A4 specific fluorescence substrate DBF was investigated in addition to 7-BQ.

TABLE 1
Detection of Basal and inducible CYP3A4 mRNA

| | | | | CYP3A4 | CYP3A7 | Cyp3a11 | mβ-actin |
|---|---|---|---|---|---|---|---|
| Tissue | Mouse # | Mouse line | Treatment | Ct value | Ct value | Ct value | Ct value |
| Liver | 4 | hCYP3A4/3A7_Cyp3a KO | Corn oil | 23 | | | 21 |
| | 10 | hCYP3A4/3A7_Cyp3a KO | PCN | 17 | 28 | | 21 |
| | 11 | hCYP3A4/3A7_Cyp3a KO | PCN | 18 | 30 | | 21 |
| | 5 | hCYP3A4_Cyp3a KO | Corn oil | 26 | | | 20 |
| | 12 | hCYP3A4_Cyp3a KO | PCN | 19 | | | 20 |
| | 13 | hCYP3A4_Cyp3a KO | PCN | 20 | | | 20 |
| | 6 | Cyp3a KO | Corn oil | | | | 22 |
| | 14 | Cyp3a KO | PCN | | | | 21 |
| | 15 | Cyp3a KO | PCN | | | | 20 |
| | 1 | WT | Corn oil | | | 19 | 21 |
| | 2 | WT | Corn oil | | | 19 | 21 |
| | 3 | WT | Corn oil | | | 19 | 21 |
| | 7 | WT | PCN | | | 16 | 20 |
| | 8 | WT | PCN | | | 15 | 20 |
| | 9 | WT | PCN | | | 15 | 20 |
| Duodenum | 4 | hCYP3A4/3A7_Cyp3a KO | Corn oil | 22 | | | 19 |
| | 10 | hCYP3A4/3A7_Cyp3a KO | PCN | 20 | | | 19 |
| | 11 | hCYP3A4/3A7_Cyp3a KO | PCN | 21 | | | 18 |
| | 5 | hCYP3A4_Cyp3a KO | Corn oil | 24 | | | 19 |
| | 12 | hCYP3A4_Cyp3a KO | PCN | 23 | | | 19 |
| | 13 | hCYP3A4_Cyp3a KO | PCN | 23 | | | 19 |
| | 6 | Cyp3a KO | Corn oil | | | | 19 |
| | 14 | Cyp3a KO | PCN | | | | 18 |
| | 15 | Cyp3a KO | PCN | | | | 18 |
| | 1 | WT | Corn oil | | | 20 | 18 |
| | 2 | WT | Corn oil | | | 22 | 19 |
| | 3 | WT | Corn oil | | | 20 | 18 |
| | 7 | WT | PCN | | | 18 | 19 |
| | 8 | WT | PCN | | | 19 | 18 |
| | 9 | WT | PCN | | | 18 | 18 |

Low basal CYP3A4 mRNA was identified, however this did not translate into protein. PCN-induced CYP3A4 protein expression was identified in this line which was comparable to humans. CYP3A4 is catalytically active in the hCYP3A4_Cyp3a KO mice relative to Cyp3a KO mice. These observations indicate that the CYP3A4 protein expressed in the hCYP3A4_Cyp3a KO mouse line is functional. CYP3A4 protein/mRNA but not CYP3A7 was identified suggesting a new utility of this model in the developmental regulation of CYP3As. CYP3A4 is highly catalytically active in the hCYP3A4/3A7_Cyp3a KO mice relative to Cyp3a KO mice. In vitro metabolism studies have revealed, Cyp3 a proteins in the mouse result in much higher levels of murine-specific metabolites compared to humans which has unfavourable toxicological implications. These observations indicate that the hCYP3A4/3A7_Cyp3a KO mouse line is functional.

### Body & Liver Weights

Further studies using 6 C57BL/6J mice (obtained from Harlan (UK)), 3 hCYP3A4/3A7_Cyp3a KO mice, 3 hCYP3A4_Cyp3a KO mice and 3 Cyp3a KO mice (supplied by TaconicArtemis, Germany) were performed. All animals used were males. Upon arrival the mice were housed on sawdust in solid-bottom polypropylene cages. No environmental enhancing materials were used during treatment.

In the animal room the environment was controlled to provide conditions required by the Home Office for accommodation and husbandry of rodents. The temperature was maintained within a range of 19-23° C. and relative humidity within a range of 40-70%. There was a nominal 14-15 air changes per hour. Twelve-hour periods of light were cycled with twelve-hour periods of darkness. For this study no special arrangement of cages was used. The mice were allowed to acclimatize for a minimum of five days following arrival at the test facility.

The animals were uniquely numbered, by ear-punch or tail marking, and allocated to groups, as shown in Table 2. An experiment card was placed on each cage and showed the project license code, treatment given, study number, sex and individual numbers of the mice within.

TABLE 2
Transgenic mouse allocation

| Mouse | | Artemis | | |
|---|---|---|---|---|
| # | Mouse line | mouse # | Gender | DOB |
| 4 | hCYP3A4/3A7_Cyp3aKO | 237197 | M | 19 Aug. 2008 |
| 5 | hCYP3A4_Cyp3aKO | 234444 | M | 12 Jul. 2008 |
| 6 | Cyp3aKO | 230453 | M | 30 Apr. 2008 |
| 10 | hCYP3A4/3A7_Cyp3aKO | 237198 | M | 19 Aug. 2008 |
| 11 | hCYP3A4/3A7_Cyp3aKO | 237199 | M | 19 Aug. 2008 |
| 12 | hCYP3A4_Cyp3aKO | 237186 | M | 26 Aug. 2008 |
| 13 | hCYP3A4_Cyp3aKO | 237187 | M | 26 Aug. 2008 |
| 14 | Cyp3aKO | 230454 | M | 30 Apr. 2008 |
| 15 | Cyp3aKO | 231969 | M | 06 Jun. 2008 |

Prior to the start of the study, all mice were observed to ensure that they were physically normal and that they exhibit normal activity. Only mice exhibiting normal behaviour were accepted for the study. Any clinical abnormalities observed in individual animals were recorded in the study diary. A general assessment of condition was recorded in the study diary.

The mice received either corn oil (vehicle) or PCN 100 mg/kg, daily, for 2 days by intraperitoneal (IP) injection according to the experimental design described in Table 3. Dosing solutions were prepared at CXR Biosciences on the day of dosing by adding the vehicle (corn oil) to the requisite quantity of the PCN. The concentration of PCN was the concentration of supplied chemical, without any correction for purity. Excess dosing solution was stored at approximately 2-8° C. for possible future analysis. The volume of dosing solution was 10 mL/kg bodyweight. Approximately 24 h after the second dose, the mice were euthanized using a rising concentration of CO₂. Blood was collected at termination by cardiac puncture into lithium/heparin coacted tubes for plasma preparation.

TABLE 3
Experimental design

| | | | | Dose | | |
|---|---|---|---|---|---|---|
| Grp | Mouse # | Mouse Line | Compound | (mg/kg) | Gender | Route |
| 1 | 1-3 | C57BL/6J | Corn oil | NA | M | IP |
| 2 | 4 | hCYP3A4/3A7_Cyp3a KO | Corn oil | NA | M | IP |
| 3 | 5 | hCYP3A4_Cyp3a KO | Corn oil | NA | M | IP |
| 4 | 6 | Cyp3a KO | Corn oil | NA | M | IP |
| 5 | 7-9 | C57BL/6J | PCN | 100 | M | IP |
| 6 | 10-11 | hCYP3A4/3A7_Cyp3a KO | PCN | 100 | M | IP |
| 7 | 12-13 | hCYP3A4_Cyp3a KO | PCN | 100 | M | IP |
| 8 | 14-15 | Cyp3a KO | PCN | 100 | M | IP |

The body weight of each mouse was recorded at the start of the study and immediately prior to termination. Bodyweights were recorded electronically or manually and records of these weights were stored in the study file.

In order to weigh the liver, the gall bladder was removed, and then the liver was removed and weighed. Two samples of liver (approximately 5 mm³) were immediately flash frozen in a cryovial in liquid nitrogen then stored at approximately −70° C. for RNA analysis. The remaining liver was weighed and immediately used for subcellular fractionation to homogenates and microsomes.

The liver/body weight ratio of the C57BL/6J mice significantly (P<0.001) increased as a result of PCN administration as shown in Table 4. The liver/body weight ratios of the control and treated transgenic mice could not be statistically compared as there was only one transgenic animal in each control group. All treated transgenic mice showed a decreased liver/body weight ratio compared to the treated wild type, although only in the Cyp3a KO group was this decrease statistically significant (P<0.05).

TABLE 4
Body and liver weights

| | | | Dose | Liver | Body | Liver per body |
|---|---|---|---|---|---|---|
| Mouse # | Mouse Line | Compound | (mg/kg) | weight, g | weight, g | weight, % |
| 1-3 | C57BL/6J | Corn oil | NA | 0.82 ± 0.03 | 18.80 ± 0.69 | 4.35 ± 0.07 |
| | | | | | | 100 ± 1.6† |
| | | | | | | 100 ± 1.6‡ |
| 4 | hCYP3A4/3A7_Cyp3a | Corn oil | NA | 0.81 | 0.81 | 4.62 |
| | KO | | | | | 100† |
| | | | | | | 106‡ |
| 5 | hCYP3A4_Cyp3a | Corn oil | NA | 1.08 | 1.08 | 4.65 |
| | KO | | | | | 100† |
| | | | | | | 107‡ |
| 6 | Cyp3a KO | Corn oil | NA | 0.93 | 0.93 | 4.19 |
| | | | | | | 100† |
| | | | | | | 96‡ |
| 7-9 | C57BL/6J | PCN | 100 | 1.10 ± 0.06 | 19.30 ± 0.82 | 5.70 ± 0.12*** |
| | | | | | | 131 ± 2.7† |
| | | | | | | 100 ± 2.1‡ |
| 10-11 | hCYP3A4/3A7_Cyp3a | PCN | 100 | 1.06 ± 0.27 | 19.15 ± 0.64 | 5.53 ± 1.20 |
| | KO | | | | | 120 ± 26.1† |
| | | | | | | 97 ± 21.1‡ |
| 12-13 | hCYP3A4_Cyp3a | PCN | 100 | 1.00 ± 0.09 | 19.00 ± 0.71 | 5.27 ± 0.26 |
| | KO | | | | | 113 ± 5.6† |
| | | | | | | 93 ± 4.6‡ |
| 14-15 | Cyp3a KO | PCN | 100 | 1.22 ± 0.15 | 24.05 ± 1.77 | 5.06 ± 0.25* |
| | | | | | | 121 ± 6.0† |
| | | | | | | 89 ± 4.4‡ |

Data are mean ± SD.
n = 3 for C57BL/6J and n = 2 for the PCN treated transgenic lines.
The liver/body weight ratios were compared with an unpaired t test (two tailed P values).
†percentage of control group of the same strain
‡percentage of C57BL/6J from the same treatment group
*statistically significant compared to C57BL/6J control group
*statistically significant compared to C57BL/6J group treated with PCN

### Plasma Clinical Chemistry

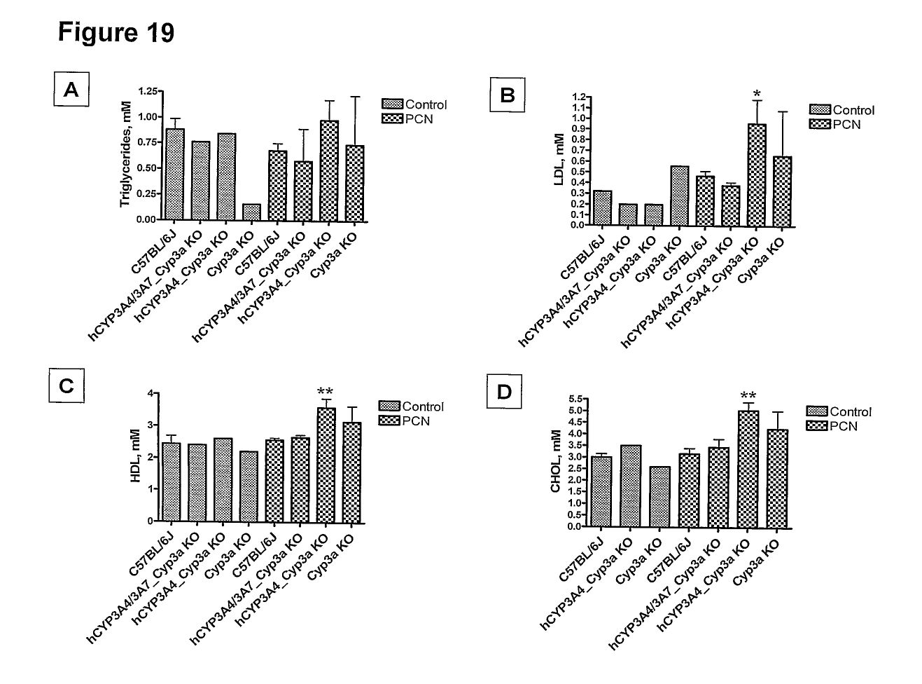

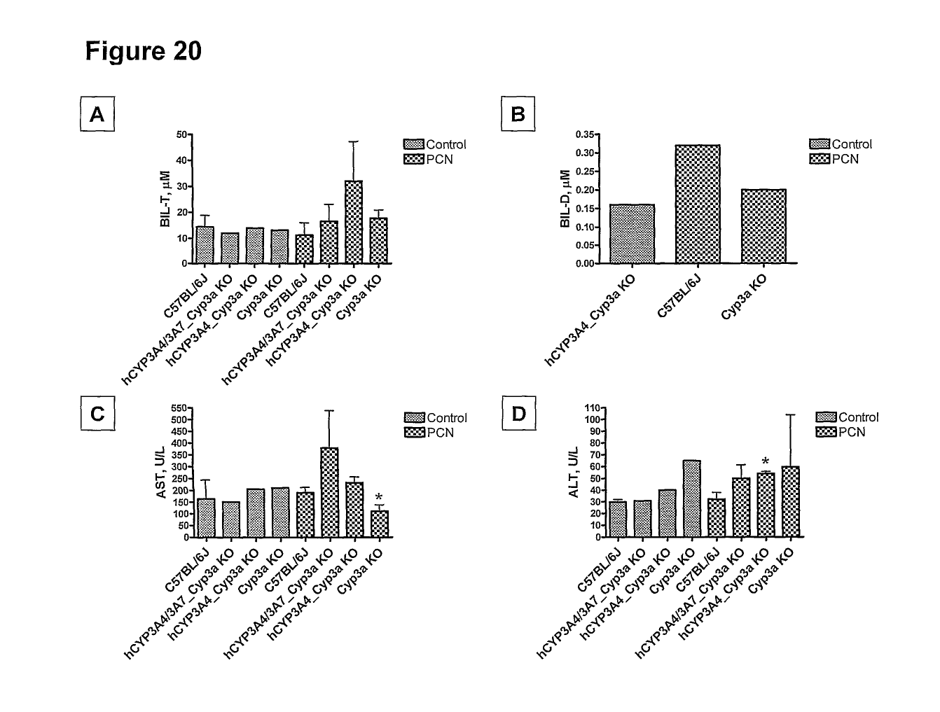

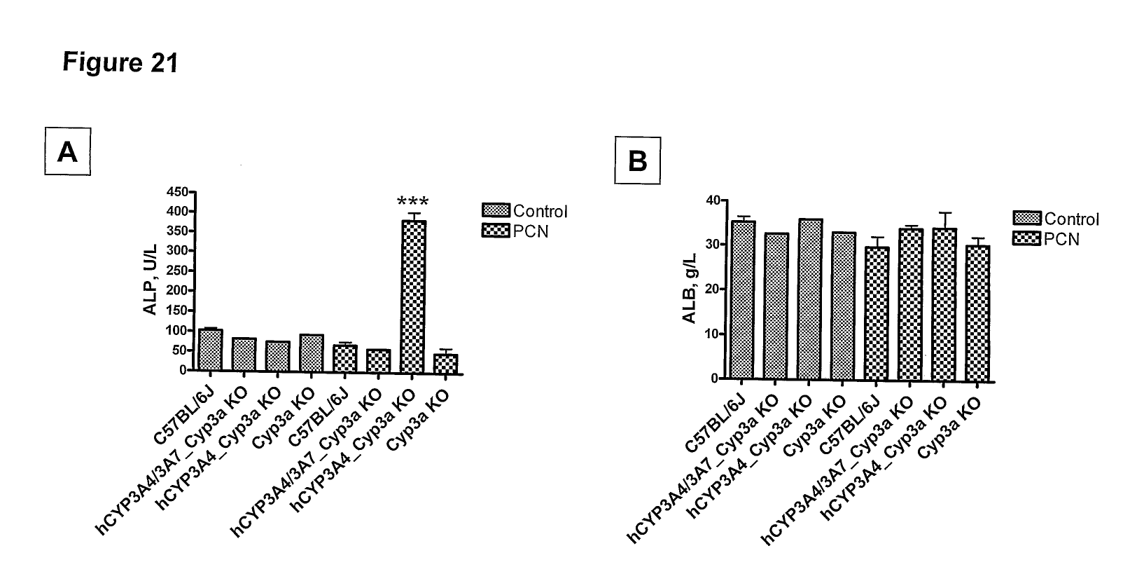

Plasma samples were produced by removing red blood cells by centrifugation (2,000-3,000 rpm for 10 min at 8-10° C.). The supernatant (plasma) was stored on ice prior to clinical chemistry analysis. The pellet was discarded. Plasma samples from all animals were analysed for triglycerides, alanine aminotransferase, alkaline phosphatase, aspartate aminotransferase, albumin, cholesterol, bilirubin (total and direct), high and low density lipoproteins using the COBAS Integra 400+ (Roche), and the results are shown in FIGS. 19-21. Plasma samples from PCN treated hCYP3A4_Cyp3a KO mice demonstrated statistically significant increases in the level of cholesterol, low and high density lipoproteins, alanine transferase and alkaline phosphatase compared to the samples from treated C57BL/6J mice. The biological significance of this increase will have to be investigated using larger group sizes. The values of plasma clinical chemistry parameters for all other samples fell within the known normal range for untreated C57BL/6J mice. Table 5 shows the range of plasma clinical chemistry parameters of the untreated C57BL/6J mice.

There was insufficient plasma from mouse 1 (control C57BL/6J) and mouse 4 (control hCYP3A4_Cyp3a KO) to perform cholesterol analysis.

Direct bilirubin in all mice apart from mouse 5 (control hCYP3A4_Cyp3a KO), mouse 8 (PCN treated C57BL/6J) and mouse 14 (PCN treated Cyp3a KO) was below the limit of quantification.

TABLE 5
Normal range of selected plasma clinical chemistry in untreated wild type C57BL/6J mice

| Mouse Line | Mean | SEM | n | Max | Min | Mean | SEM | n | Max | Min | Mean | SEM | n | Max | Min |
|---|---|---|---|---|---|---|---|---|---|---|---|---|---|---|---|
| | ALP (U/L) | | | | | ALT (U/L) | | | | | AST (U/L) | | | | |
| WT | 77 | 32 | 93 | 183 | 11 | 41 | 18 | 96 | 132 | 11 | 101 | 40 | 92 | 215 | 26 |
| | Albumin (g/L) | | | | | BIL-D (μmol/L) | | | | | BIL-T (μmol/L) | | | | |
| WT | 30 | 10 | 50 | 55 | 0 | 0.07 | 0.20 | 61 | 1.00 | 0.00 | 19 | 20 | 60 | 72 | 0 |
| | Cholesterol (mmol/L) | | | | | HDL (mmol/L) | | | | | LDL (mmol/L) | | | | |
| WT | 2.54 | 0.79 | 61 | 4.20 | 0.10 | 2.13 | 0.66 | 55 | 3.50 | 0.00 | 0.22 | 0.13 | 53 | 0.57 | 0.00 |

| | Triglycerides (mmol/L) | | | | |
|---|---|---|---|---|---|
| Mouse Line | Mean | SEM | n | Max | Min |
| WT | 1.75 | 0.61 | 60 | 3.10 | 0.12 |

Values represent Mean ± SEM for the following plasma analytes; ALP, ALT, AST, albumin, BIL-D, BIL-T, cholesterol, HDL, LDL and triglycerides.
Maximum and minimum vales are also included.
Data was generated by collating plasma clinical chemistry values across multiple studies generated using the COBAS 400+ integra (Roche).
There was insufficient plasma from mouse 1 (control C57BL/6J) and mouse 4 (control hCYP3A4_Cyp3a KO) to perform cholesterol analysis.
Direct bilirubin in all mice apart from mouse 5 (control hCYP3A4_Cyp3a KO), mouse 8 (PCN treated C57BL/6J) and mouse 14 (PCN treated Cyp3a KO) was below the limit of quantification.

### Western Blot Analysis of Liver and Intestinal Microsomes for CYP3A4

The duodenum (first 10 cm from the base of the stomach) was removed and flushed with ice cold PBS containing a protease inhibitor cocktail (Roche). The first 2 cm was but and placed into a 2 ml cryvial containing 1 ml of TRIZOL (Sigma), flash frozen immediately, and then stored at approximately −70° C. for Taqman® analysis. The remainder of the duodenum was placed in a 1 ml cryovial and flash frozen immediately, and then stored at approximately −70°.

Liver microsomes were produced by preparing subcellular fractions from fresh livers. The livers were processed as described above to homogenates and microsomes. Aliquots from liver samples were stored at approximately −70° prior to analysis.

Frozen small intestines were homogenised in SET with protease cocktail inhibitor (Roche) and PMSF (mM) using a Polytron homogeniser. The homogenates were subjected to subcellular fractionation as described above. The microsomal fractions were stored at approximately −70° prior to analysis.

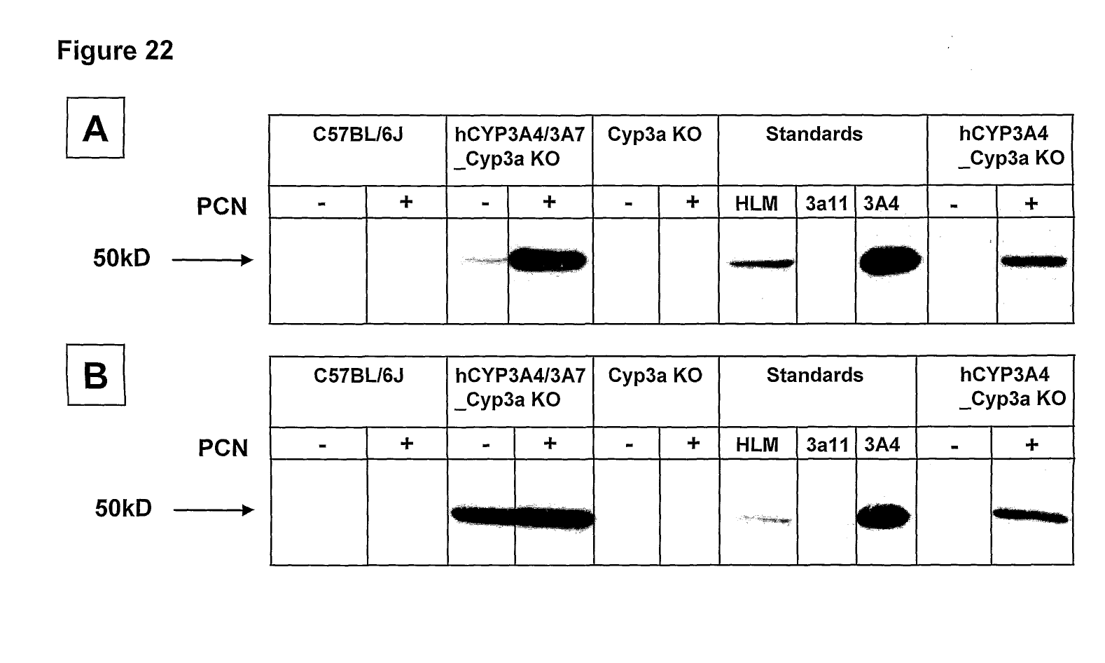

Liver microsomes from C57BL/6J, hCYP3A4/3A7_Cyp3a KO, hCYP3A4_Cyp3a KO and Cyp3a KO mice were analysed by Western blotting using an antibody specific to CYP3A4 and the results are shown in FIG. 22. There was a clear protein band for CYP3A4 on the Western blot of liver and intestinal microsomes from vehicle treated hCYP3A4/3A7_Cyp3a KO mice. In liver microsomes from control hCYP3A4_Cyp3a KO mice, the protein level of CYP3A4 was below the limit of detection. However there was a low intensity CYP3A4 protein band on the immunoblot of the intestinal microsomes from this mouse strain. Administration of PCN resulted in strong upregulation of liver CYP3A4 in the humanised mice. In the intestinal samples this upregulation was less pronounced. The level of CYP3A4 protein in microsomes from C57BL/6J and Cyp3a KO animals was below the limit of detection.

### Western Blot Analysis of Liver and Intestinal Microsomes for CYP3A and Cyp3a

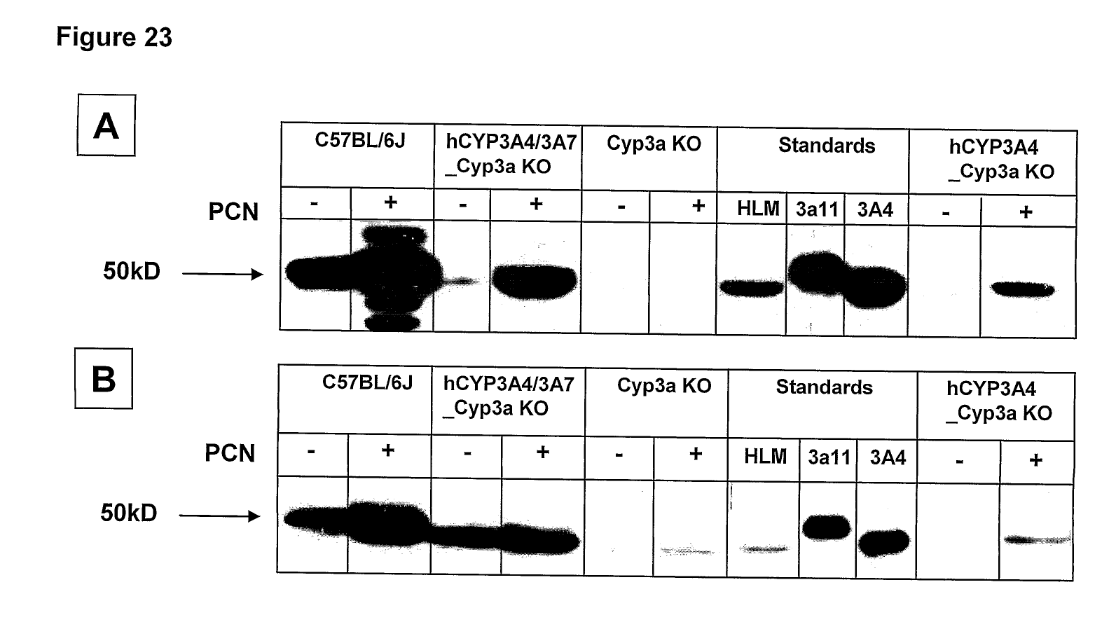

Western blots of liver microsomes from C57BL/6J, hCYP3A4/3A7_Cyp3a KO, hCYP3A4_Cyp3a KO and Cyp3a KO mice were analysed using an antibody which has an affinity to both human CYP3A and mouse Cyp3a isoforms and the results are shown in FIG. 23. Significantly lower levels of protein expression were detected in the livers of mice from both humanised transgenic lines compared to wild type. The lower intensity band observed in humanised transgenic mouse liver should represent CYP3A4 expression only because these mice are null for the Cyp3a family. The difference was less pronounced following PCN treatment, with the hCYP3A4/3A7_Cyp3a KO mouse sample having higher CYP3A/Cyp3a protein expression compared to that from hCYP3A4_Cyp3a KO mouse. In intestinal microsomes there was a similar level of CYP3A/Cyp3a protein in both wild type and hCYP3A4/3A7_Cyp3a KO mice. The intestinal samples from hCYP3A4_Cyp3a KO mice produced very low intensity bands, similar to those from Cyp3a KO mice.

### In Vitro Oxidation of 7-Benzyloxyquinoline (7-BQ) by Liver and Intestinal Microsomes

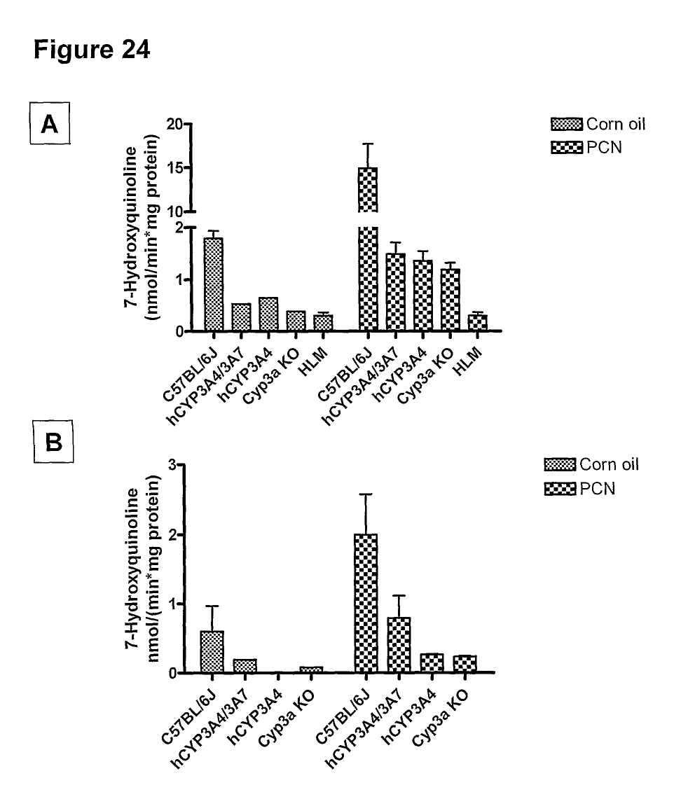

There was a marked decrease in 7-BQ oxidation by microsomes from Cyp3a KO mice compared to the wild type animals as shown in FIG. 24. Although humanised lines demonstrated some recovery of activity relative to the Cyp3a KO strain, this activation was small and not statistically significant. Moreover, pooled human liver also showed a low reaction rate, suggesting that 7-BQ is a better substrate for murine Cyp3a than for human CYP3A4.

### In Vitro DBF Oxidation by Liver and Intestinal Microsomes

DBF (2 μM) was incubated with 5 μL liver or 25 μA, intestinal microsomes in 50 mM HEPES buffer pH 7.4 (15 mM MgCl2, 0.1 mM EDTA) at 37° C. for approximately 50 sec before the reaction was started by addition of 20 μL NADPH (42 mg/mL). The total reaction volume was 1 mL. Fluorescein fluorescence was recorded using an F-4500 fluorescence spectrophotometer (Hitachi), excitation 485 nm and emission 538 nm. Fluorescein standard (10 μL, 25 μM) was injected into the reaction cuvette approximately 150 sec after the addition of NADPH. Slopes of the time course of the product accumulation were calculated using FL-Solution 2.0 (Hitachi).

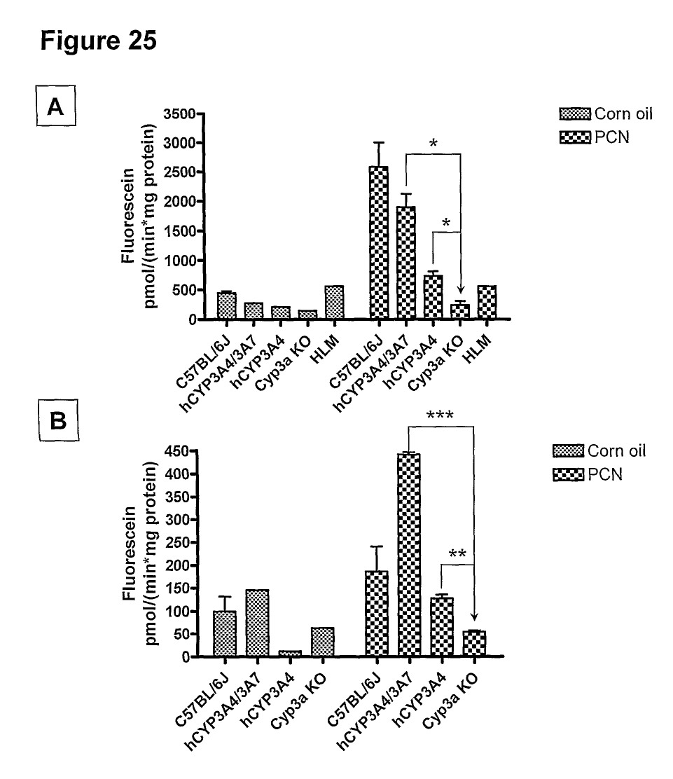

Both liver and intestinal samples from the untreated humanised mice demonstrated little increase in DBF oxidation activity compared to Cyp3a KO. However, the difference was significantly more pronounced in samples from the PCN treated groups as shown in FIG. 25. The reaction rate was higher in hCYP3A4/3A7_Cyp3a KO microsomes compared to that from hCYP3A4_Cyp3a KO animals. This correlated with the Western blotting data.

### In Vitro Oxidation of a Clinically Relevant Substrate of CYP3A4 by Liver and Intestinal Microsomes

Triazolam was selected as a clinically relevant substrate of CYP3A4. It is currently used for treatment of insomnia (website of American Society of Health-System Pharmacists). It was also shown to be a selective substrate not only for human CYP3A4 but also for murine cytochromes P450 from the Cyp3a subfamily (Perloff et al., 2000).

Triazolam (50 μM) was incubated with microsomes (2.5 μL liver microsomes or 6 μL intestinal microsomes) and NADPH (1.3 mM) in 50 mM HEPES buffer pH 7.4 (15 mM MgCl2, 0.1 mM EDTA) at 37° C. The total reaction volume was 200 μL. After 15 min, the reaction was stopped by taking an aliquot (80 μL) of the reaction mixture and adding it to an equal volume of ice-cold acetonitrile. Samples were centrifuged at approximately 13,000 g for 10 minutes and α-hydroxytriazolam concentration in the supernatant was determined by LC-MS/MS (Tables 6-7). 20 μL of the supernatant was injected onto the LC-MS/MS system.

TABLE 6
Multiple reaction monitoring parameters
of triazolam and α-hydroxytriazolam

| | Ion | Parent | Collision | Cone | Collision |
|---|---|---|---|---|---|
| Compound | mode | ion | ion | Voltage (V) | energy (eV) |
| *Triazolam | ES+ | 343.44 | 308.28 | 25 | 29 |
| α-Hydroxy- | ES+ | 359.27 | 176.16 | 25 | 31 |
| triazolam | | | | | |

Run Time: 7.5 minutes.
Ion source: electrospray positive ion mode
Detector: Micromass Quattro Micro mass spectrometer

TABLE 7
HPLC parameters for separation of
triazolam and α-hydroxytriazolam

| Time | | | Flow rate | |
|---|---|---|---|---|
| (min) | % A | % B | (ml/min) | Curve |
| 0 | 85 | 15 | 0.4 | 1 |
| 1.0 | 85 | 15 | 0.4 | 6 |
| 2.0 | 25 | 75 | 0.4 | 6 |
| 4.0 | 10 | 90 | 0.4 | 6 |
| 4.5 | 5 | 95 | 0.4 | 6 |
| 5.0 | 85 | 15 | 0.4 | 2 |
| 5.5 | 85 | 15 | 0.4 | 1 |

Column = Luna, C18, 5 μm, 150 × 2.0 mm
Solvent A = 10 mM Ammonium acetate
Solvent B = 0.1% formic acid in acetonitrile

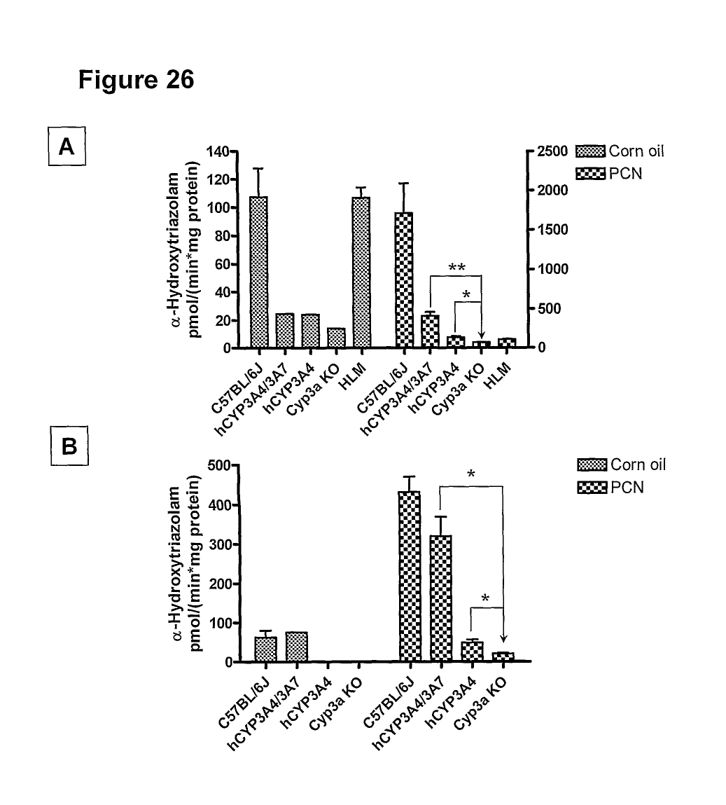

Similarly to DBF, the rate of triazolam oxidation was slightly faster in the microsomes from the control humanised mice compared to Cyp3a KO strain as shown in FIG. 26. Microsomes from induced humanised animals showed significantly higher activity, and samples from hCYP3A4/3A7_Cyp3a KO mice were more active than those from the hCYP3A4_Cyp3a KO line.

### RT-PCR and Sequencing of CYP3A4 and CYP3A7

The following oligonucleotides specific for CYP3A4 and CYP3A7 were used during the RT-PCR reactions:

3A4_F_B gct gaa agg aag act cag agg Tm: 59.8
3A4_R_B ggc aca gat ttc ttg aag agc Tm: 57.9
3A7_F gac tca gag gag aga gat aag g Tm: 60.3
3A7_R gca aac cag aag tcc tta ggg Tm: 59.8

Total RNA was prepared from liver tissue of humanised (hCYP3A4/3A7_Cyp3a KO (mouse 4) and hCYP3A4_Cyp3a KO (mouse 5)) and wild type C57BL/6J (mouse 1) mice using an RNeasy kit (QIAGEN, Cat No. 74104) according to the manufacturer's instructions, and purified using RNeasy kit (QIAGEN).

RT-PCR was conducted using a Superscript III One-Step RT-PCR Platinum Taq HiFi Kit (Invitrogen Corp. Cat. No. 12574-030) according to the manufacturer's protocol. The products of RT-PCR were separated by electrophoresis on an agarose gel. A DNA fragment of the predicted size was extracted from agarose gel, and then cloned into vector pCR4-TOPO using a TOPO TA Cloning kit for Sequencing (Invitrogen Corp. Cat. no. K4575-01).

One step RT-PCR set up (for both CYP3A4 and CYP3A7):

cDNA synthesis:

### 48° C. 30 min

### 94° C. 2 min

PCR amplification: 40 cycles of:

### 94° C. 30 sec

### 54° C. 30 sec

### 68° C. 2 min

### Final Extension:

### 68° C. 5 min

Sequence analysis was performed by: Lark Technologies, Ltd., A Genaissance Company, Hope End, Takeley, Essex CM22 6TA.

Alignments were performed using Vector NTI 8 Software, utilising Contig express and Align-X and T-COFFEE http://www.ch.embnet.org/software/TCoffee.html

### Characterisation of Human CYP3A4 Transcript

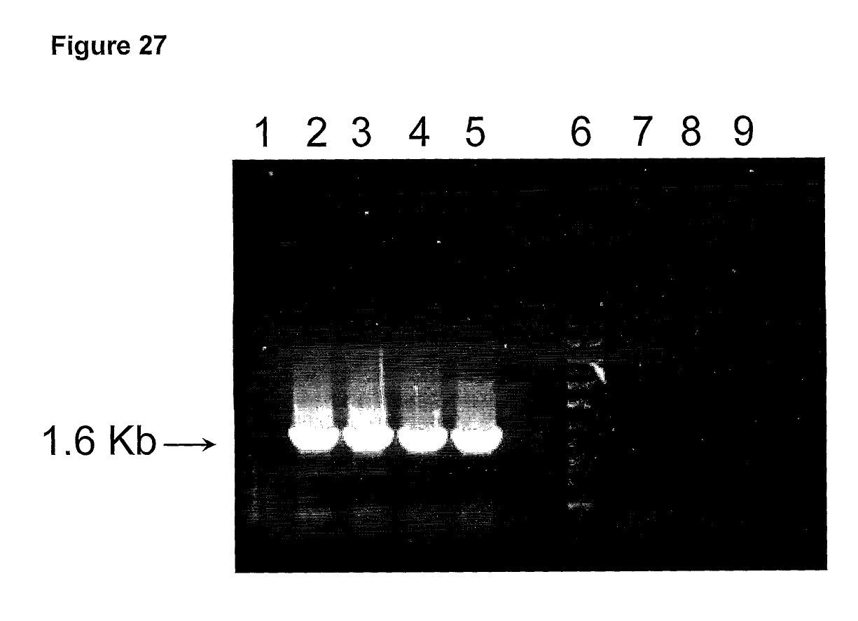

Total hepatic RNA samples isolated from vehicle-treated wild type (mouse 1) and humanised (mice 4-5) animals were analysed by RT-PCR using primers 3A4_F_B and 3A4_R_B A DNA fragment of the predicted size (˜1.6 Kb) was observed as shown in FIG. 27. The DNA fragments from mice 4 and 5 were extracted from the agarose gel and separately cloned into the pCR4/TOPO vector.

Two selected clones were analysed by sequencing. Sequence alignments of these clones with the CYP3A4 cDNA used in the targeting vector (TaconicArtemis) showed that the cloned CYP3A4 was derived from a full-length transcript.

### Characterisation of Human CYP3A7 Transcript

Total hepatic RNA sample isolated from humanised mouse 4 (hCYP3A4/3A7_Cyp3a KO) was analysed by RT-PCR using primers 3A7_F and 3A7_R. No DNA product was observed in either wild type (mouse 1) or humanised (mouse 4) mice as shown in FIG. 27.

TaqMan® Analysis of CYP3A4, CYP3A7 and Cyp3a11 mRNA Expression

Estimation of CYP3A4 and CYP3A7 mRNA levels was performed by Q-PCR analysis using CYP3A4 and CYP3A7 specific primers. β-Actin was used as a reference gene. The Q-PCR analysis of the liver and intestinal samples is summarised in Table 8.

TABLE 8
Average threshold cycle (Ct) and delta Ct (dCt) values for CYP3A4 (3A4),
CYP3A7 (3A7) and β-actin from the liver and intestinal samples.

| | | Average Ct | | | dCt | |
|---|---|---|---|---|---|---|
| Mouse strain (animal #) | Treatment | 3A4 | 3A7 | β-actin | 3A4 | 3A7 |
| Liver | | | | | | |
| C57BL/6J(1) | Corn oil | 33 | NQ | 21 | 11 | NQ |
| C57BL/6J(2) | Corn oil | 30 | NQ | 21 | 9 | NQ |
| C57BL/6J(3) | Corn oil | 30 | 40 | 21 | 9 | 19 |
| hCYP3A4/3A7_Cyp3a KO (4) | Corn oil | 23 | 37 | 21 | 2 | 16 |
| hCYP3A4_Cyp3a KO (5) | Corn oil | 26 | 35 | 20 | 6 | 15 |
| Cyp3a KO (6) | Corn oil | 38 | NQ | 22 | 16 | NQ |
| C57BL/6J (7) | PCN | 30 | 38 | 20 | 10 | 18 |
| C57BL/6J (8) | PCN | 32 | NQ | 20 | 12 | NQ |
| C57BL/6J (9) | PCN | 32 | NQ | 20 | 12 | NQ |
| hCYP3A4/3A7_Cyp3a KO | PCN | 17 | 28 | 21 | −3 | 8 |
| (10) | | | | | | |
| hCYP3A4/3A7_Cyp3a KO | PCN | 18 | 30 | 21 | −2 | 9 |
| (11) | | | | | | |
| hCYP3A4_Cyp3a KO (12) | PCN | 19 | NQ | 20 | −1 | NQ |
| hCYP3A4_Cyp3a KO (13) | PCN | 20 | 38 | 20 | 0 | 18 |
| Cyp3a KO (14) | PCN | 38 | NQ | 21 | 17 | NQ |
| Cyp3a KO (15) | PCN | 31 | NQ | 20 | 11 | NQ |
| Small intestine | | | | | | |
| C57BL/6J(1) | Corn oil | 32 | 37 | 18 | 13 | 18 |
| C57BL/6J(2) | Corn oil | 39 | NQ | NQ | 20 | 19 |
| C57BL/6J(3) | Corn oil | 37 | NQ | NQ | 19 | 18 |
| hCYP3A4/3A7_Cyp3a KO (4) | Corn oil | 22 | 39 | 20 | 3 | 19 |
| hCYP3A4_Cyp3a KO (5) | Corn oil | 24 | NQ | NQ | 5 | 19 |
| Cyp3a KO (6) | Corn oil | NQ | NQ | NQ | NQ | 19 |
| C57BL/6J (7) | PCN | 35 | NQ | NQ | 17 | 19 |
| C57BL/6J (8) | PCN | 37 | NQ | NQ | 19 | 18 |
| C57BL/6J (9) | PCN | NQ | NQ | NQ | NQ | 18 |
| hCYP3A4/3A7_Cyp3a KO | PCN | 20 | 39 | 20 | 1 | 19 |
| (10) | | | | | | |
| hCYP3A4/3A7_Cyp3a KO | PCN | 21 | 38 | 20 | 2 | 18 |
| (11) | | | | | | |
| hCYP3A4_Cyp3a KO (12) | PCN | 23 | NQ | NQ | 4 | 19 |
| hCYP3A4_Cyp3a KO (13) | PCN | 23 | NQ | NQ | 5 | 19 |
| Cyp3a KO (14) | PCN | 39 | NQ | NQ | 21 | 18 |
| Cyp3a KO (15) | PCN | 39 | NQ | NQ | 21 | 18 |

Each sample was analysed in triplicates for the reference and target genes.
Amplification curves were processed using Sequence Detection Software 1.2.3 (Applied Biosystems).
NQ—not quantifiable (reaction curves do not allow the quantification of Ct value)

For each target gene a reaction with the lowest dCt value was identified and that dCt value was subtracted from all other dCt, giving so-called ddCt (ddCt of the sample with the lowest dCt (endogenous reference) equals 0). Finally, the normalised relative amount of target gene (RQ) was calculated using the following formula: RQ=(2̂(−ddCt))*100 as shown in Table 9.

TABLE 9
Relative quantification (RQ) values obtained from dCt as described above.

| | | RQ, % | | |
|---|---|---|---|---|
| Mouse strain (animal #) | Treatment | CYP3A4 | CYP3A7 | Cyp3a11 |
| Liver | | | | |
| C57BL/6J(1) | Corn oil | 0.00 | NQ | 11.35 |
| C57BL/6J(2) | Corn oil | 0.00 | NQ | 10.81 |
| C57BL/6J(3) | Corn oil | 0.00 | 0.00 | 10.28 |
| hCYP3A4/3A7_Cyp3a KO (4) | Corn oil | 0.94 | 0.07 | 0.00 |
| hCYP3A4_Cyp3a KO (5) | Corn oil | 0.03 | 0.12 | 0.00 |
| Cyp3a KO (6) | Corn oil | 0.00 | NQ | 0.00 |
| C57BL/6J (7) | PCN | 0.00 | 0.01 | 116.20 |
| C57BL/6J (8) | PCN | 0.00 | NQ | 203.23 |
| C57BL/6J (9) | PCN | 0.00 | NQ | 135.41 |
| hCYP3A4/3A7_Cyp3a KO (10) | PCN | 219.27 | 283.53 | 0.00 |
| hCYP3A4/3A7_Cyp3a KO (11) | PCN | 86.08 | 55.00 | 0.00 |
| hCYP3A4_Cyp3a KO (12) | PCN | 20.11 | NQ | 0.00 |
| hCYP3A4_Cyp3a KO (13) | PCN | 15.06 | 0.01 | 0.00 |
| Cyp3a KO (14) | PCN | 0.00 | NQ | 0.00 |
| Cyp3a KO (15) | PCN | 0.00 | NQ | 0.04 |
| Small intestine | | | | |
| C57BL/6J(1) | Corn oil | 0.00 | ND | 52.90 |
| C57BL/6J(2) | Corn oil | 0.00 | ND | 23.18 |
| C57BL/6J(3) | Corn oil | 0.00 | ND | 28.63 |
| hCYP3A4/3A7_Cyp3a KO (4) | Corn oil | 29.59 | ND | 0.00 |
| hCYP3A4_Cyp3a KO (5) | Corn oil | 3.62 | ND | 0.00 |
| Cyp3a KO (6) | Corn oil | NQ | ND | 0.00 |
| C57BL/6J (7) | PCN | 0.00 | ND | 256.04 |
| C57BL/6J (8) | PCN | 0.00 | ND | 169.41 |
| C57BL/6J (9) | PCN | NQ | ND | 250.72 |
| hCYP3A4/3A7_Cyp3a KO (10) | PCN | 154.98 | ND | 0.00 |
| hCYP3A4/3A7_Cyp3a KO (11) | PCN | 62.57 | ND | 0.00 |
| hCYP3A4_Cyp3a KO (12) | PCN | 9.31 | ND | 0.02 |
| hCYP3A4_Cyp3a KO (13) | PCN | 5.53 | ND | 0.00 |
| Cyp3a KO (14) | PCN | 0.00 | ND | 0.00 |
| Cyp3a KO (15) | PCN | 0.00 | ND | NQ |

RQ values for CYP3A7 from intestinal samples were not determined because dCt values did not indicated the presence of CYP3A7 mRNA.
NQ—non quantifiable (reaction curves did not allow the quantification of Ct value)
ND—not determined

CYP3A4 mRNA was confidently detected both in the liver and in small intestine of the control and treated humanised mice. CYP3A7 mRNA level was below the detection limit in the liver of the control hCYP3A4/3A7_Cyp3a KO mice. This data was consistent with the results from RT-PCR and sequencing of CYP3A4 and CYP3A7. However, Q-PCR analysis of the livers of treated hCYP3A4/3A7_Cyp3a KO mice indicated possible induction of CYP3A7 as a result of administration of PCN. CYP3A7 mRNA was undetectable in the intestinal samples. There was no difference in the level of Cyp3a11 mRNA between the transgenic animals (data not shown). Representation of Q-PCR data as relative quantification (RQ) confirmed the inductive effect of PCN.

Constitutive expression of CYP3A4 protein was detected in liver microsomes of male hCYP3A4/3A7 mice using a CYP3A4 specific antibody. However the expression level of this enzyme was markedly lower than that of murine Cyp3a according to the results of the immunoblot for CYP3A/Cyp3a protein. Intestinal microsomes of C57BL/6J and hCYP3A4/3A7_Cyp3a KO mouse lines demonstrated similar expression of CYP3A/Cyp3a protein. The constitutive expression of hepatic CYP3A4 in hCYP3A4_Cyp3a KO mice was below the detection limit of Western blotting and the intestinal sample from this strain demonstrated a very low intensity band of CYP3A4. The immunoblot data were generally consistent with the activities in oxidation of the CYP3A4 specific substrates, although any statistical comparison was not possible as only one animal from each transgenic strain was available.

Treatment with PCN resulted in strong induction of hepatic and intestinal CYP3A4 in both humanised lines. The expression of CYP3A/Cyp3a in the treated C57BL/6J and hCYP3A4/3A7_Cyp3a KO animals was comparable whilst that in hCYP3A4_Cyp3a KO mice was markedly lower. This was in agreement with CYP3A4 specific enzyme activities measured using DBF and triazolam. However, when 7-BQ was used as the substrate no increase in activity was observed in CYP3A4_Cyp3a KO and CYP3A4/3A7_Cyp3a KO mouse liver in response to PCN. One possible explanation for this observation is that 7-BQ is a better substrate for murine Cyp3a than for human CYP3A4, especially given that pooled human liver also showed a low reaction rate.

CYP3A4 mRNA was detected in the liver and small intestine of both humanised lines. Reverse transcription and subsequent sequencing demonstrated that the cDNA was derived from a full-length CYP3A4 transcript.

CYP3A7 mRNA was undetectable in samples from the control animals. CYP3A7 is the major CYP3A isoform expressed in human foetal liver, and undergoes a developmental switch in the first week of postnatal life, with CYP3A7 virtually disappearing concomitant with transcriptional activation of the CYP3A4 gene (Stevens et al., 2003; Hines, 2008). A similar developmental switch has also been observed in the mouse (Cyp3a16 to Cyp3a11) (Stevens et al., 2003). The mice used in this experiment were 9-15 weeks old and therefore, the expression of CYP3A7 might be switched to the expression of CYP3A4. Interestingly, some CYP3A7 mRNA was detected in the livers of PCN treated hCYP3A4/CYP3A7_Cyp3a KO mice. This has not been observed previously. Indeed, down-regulation of the CYP3A7 as a result of treatment with CYP3A4 inducers has been reported (Krusekopf et al., 2003; Hara et al., 2004).

### Example 4

### Generation of a Cyp2c Cluster Knockout Cell Line

### Construction of Cyp2c Cluster Targeting Vectors

Cyp2c cluster targeting vectors were produced as described in Example 1 for Cyp3a cluster targeting vectors.

### Cre-Mediated In Vitro Deletion of the Cyp2c Cluster in Double Targeted ES Cell

Cre-mediated deletion of the Cyp2c cluster in double targeted ES cells was performed as described in Example 1 for Cre-mediated Cyp3a cluster deletion.

1. A method of introducing a heterologous replacement gene sequence into a host cell to replace an endogenous host gene target sequence, the method comprising:
a) incorporating a pair of identical site-specific recombinase target (RT) sites of type I into the same allele of a host chromosome in separate homologous recombination steps such that the endogenous host gene target sequence that is to be replaced is flanked on each side by said identical type I RT sites; wherein one of the identical type I RT sites is flanked by a type II RT site positioned proximal to the type I RT site, wherein the type II RT site is different to the type I RT site such that it is heterospecific, and as such cannot interact with the type I RT site and;
b) effecting recombination between said pair of type I site-specific recombination sites such that the endogenous host gene target sequence is excised, and whereby a residual type I RT site remains in the chromosome at the excision point; and
c) bringing a heterologous replacement gene sequence into contact with the host chromosome, whereby the heterologous replacement gene sequence is flanked on one side by a type I RT site and on the other side by a type II RT site, under appropriate conditions to effect targeted site-specific recombinase mediated insertion of the heterologous replacement gene sequence into the host chromosome by effecting recombination between corresponding type I and type II site-specific recombination sites flanking the heterologous replacement gene sequence and located in the host chromosome, such that the heterologous gene sequence is introduced at the position in the host chromosome previously occupied by the host target gene.

2. The method of claim 1 wherein each of said type I RT sites incorporated into said host chromosome in step a) is constructed so as to be contiguous with one or more selectable markers.

3. The method of claim 2 wherein said one or more selectable markers are positioned so that said selectable markers lie between said mouse target sequence and said type I RT site.

4. The method of claim 1 wherein the heterologous replacement gene sequence is linked to one or more selectable markers.

5. The method of claim 4 wherein said one or more selectable markers are positioned between said type I RT site and said heterologous replacement gene sequence, and/or between said type II RT site and said heterologous replacement gene sequence.

6. The method of claim 4 wherein at least one selectable marker is positioned on either side of said heterologous replacement gene sequence.

7. The method of claim 2 wherein said selectable markers are selected from a neomycin expression cassette, a hygromycin resistance gene and a promoter-less and ATG-deficient Neomycin cassette (5′ΔNeo).

8. The method of claim 7 wherein at least one of said selectable markers is an ATG-deficient Neomycin cassette (5′ΔNeo).

9. The method of claim 8 wherein the endogenous host gene target sequence promoter and ATG remain in the host chromosome following recombination between said type I RT sites, such that upon insertion of the vector, said 5′ΔNeo becomes operatively linked to said promoter and ATG so that neomycin resistance is expressed.

10. The method of claim 1 wherein said type II RT site incorporated into the host chromosome is flanked by a type III RT site, such that said type II RT site is positioned between said type I RT site and said type III RT site, and wherein said type I RT site present in the host chromosome, which is not flanked proximally by a type II RT site is flanked by a type IV RT site, such that the type I RT site is positioned between the endogenous host gene target sequence and the type IV RT site.

11. The method of claim 9, wherein said vector contains a type III RT site and a type IV RT site located such that said type IV RT site is positioned between said type I RT site and said heterologous replacement gene sequence, and said type III RT site is positioned between said type II RT site and said heterologous replacement gene sequence.

12. The method of claim 10 wherein effecting recombination between corresponding type I and type II RT sites located on the vector and in the host chromosome to effect recombinase mediated insertion of the heterologous replacement gene sequence into the host chromosome results in positioning said one or more selection markers present on one side of said heterologous replacement gene sequence and the residual type I RT site between two type III RT sites, and said one or more selection markers present on the other side of said heterologous replacement gene sequence and the residual type II RT site between two type IV RT sites.

13. The method of claim 10 wherein effecting recombination between said two type III RT sites and between said two type IV RT sites results in excision of said one or more selectable markers and said residual type I or type II RT site on each side of said heterologous replacement gene sequence, and whereby a residual type III RT site and a residual type IV RT site remains in the chromosome at the excision point.

14. The method of claim 10 wherein none of said type I RT site, said type II RT site, said type III RT site and said type IV RT site are the same, such that each type of RT site is heterospecific with respect to each of the other types of RT sites, and as such that none of the RT sites can interact with another RT site of a different type.

15. The method of claim 1 wherein the site-specific recombination sites are chosen from loxP, lox5171, F3 and FRT.

16. The method of claim 15 wherein said type I RT sites is loxP.

17. The method of claim 15 wherein said type II RT site is lox5171.

18. The method of claim 15 wherein said type III RT site is FRT.

19. The method of claim 15 wherein said type IV RT site is F3.

20. The method of claim 1, wherein said heterologous replacement gene sequence is positioned on a vector.

21. The method of claim 20, wherein said vector is selected from a cloning vector, a BAC or a YAC.

22. The method of claim 1 wherein said recombination is performed in vivo.

23. The method of claim 22 wherein recombination is effected by expression of the corresponding site-specific recombinase from an expression plasmid.

24. The method of claim 1, wherein the host cell is a stem cell, such as an embryonic stem cell.

25. The method of claim 24 wherein said embryonic stem cell is a mammalian embryonic stem cell.

26. The method of claim 25, wherein said mammalian embryonic stem cell is a mouse embryonic stem cell.

27. The method of claim 24, wherein said embryonic stem cell is subsequently inserted into a blastocyst.

28. The method of claim 27 wherein said blastocyst is transplanted into a pseudo-pregnant mammal.

29. The method of claim 28, wherein said pseudo-pregnant mammal is a pseudo-pregnant mouse.

30. The method of claim 1 wherein said recombination step is performed in vitro.

31. The method of claim 1 wherein said heterologous replacement gene sequence is a mammalian gene sequence.

32. The method of claim 31, wherein said mammalian replacement gene sequence is a human replacement gene sequence.

33. A transgenic mammal humanised for the gene of in interest by the method of claim 1.

34. The transgenic mammal of claim 33 which is a transgenic mouse.
